(19) United States
(12) Patent Application Publication
(10) Pub. No.: US 2026/0250720 A1
(43) Pub. Date: Aug. 27, 2026

(54) METHODS FOR SELECTING A MICROBIAL STRAIN FOR INCREASED ISOPRENOL PRODUCTION USING AN ISOPRENOL BIOSENSOR

(71) Applicant: THE REGENTS OF THE UNIVERSITY OF CALIFORNIA, Oakland, CA (US)

(72) Inventors: Thomas T. ENG, El Cerrito, CA (US); Javier MENASALVAS, Berkeley, CA (US); Aindrila MUKHOPADHYAY, San Francisco, CA (US); Megan GARBER, Berkeley, CA (US)

(21) Appl. No.: 19/546,198

(22) Filed: Feb. 20, 2026

Related U.S. Application Data
(60) Provisional application No. 63/761,819, filed on Feb. 21, 2025.

(51) Int. Cl. C12P 7/18; C12N 15/78; C12Q 1/04

(52) U.S. Cl. CPC C12P 7/18; C12N 15/78; C12Q 1/04

(57) ABSTRACT

The present invention provides for a method for increasing the production of a short chain alcohol, such as isoprenol or a diol, comprising: (a) providing a microbial strain comprising a genetic circuit comprising a short chain alcohol biosensor which allows growth of the microbial strain only when the microbial strain produces the short chain alcohol is produced, (b) screening for a cell with increased short chain alcohol production, and (c) optionally culturing or growing the cell to produce the short chain alcohol.

### CROSS-REFERENCE TO RELATED APPLICATIONS

This application claims priority to U.S. Provisional Patent Application Ser. No. 63/761,819, filed Feb. 21, 2025, all of which are hereby incorporated by reference.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of the production of isoprenol.

### BACKGROUND OF THE INVENTION

Microbially-derived fuels hold the promise to lessen our dependence on fossil fuels, a primary driver of climate change. Biofuels like ethanol (generated by enhancing native yeast fermentation) are among the most well-known, but synthetic biology can produce advanced fuel molecules with superior combustion properties. Many short to medium chain alcohols have been evaluated as candidate fuels of which several can be produced using bioconversion processes. Even as high throughput strain engineering methods have facilitated the discovery and optimization of new molecules in general, biofuel molecules remain particularly challenging. Unlike dyes, plastics, or food additives, alcohols have physical properties that impede development cycles from both analytical and biological standpoints. Analytically, alcohols are colorless and volatile molecules that are difficult to measure with high throughput methods. The gold standard for alcohol quantification is with gas chromatography, a medium throughput method prone to operator fatigue and error. Biologically, many alcohols are growth inhibitory at high concentrations necessary for techno-economic viability due to their pleiotropic effect on cell physiology, placing an upper limit on heterologous production. Heterologous pathways can also lead to the accumulation of potentially toxic intermediate pathway metabolites. The additional burden in these strains often result in phenotypic drift where alcohol production is lost after several generations of growth. Unpredictable performance increases the risk when evaluating a strain for industrial scaleup. Therefore, it is crucial that new tools-specific to the challenges of screening alcohols-must be found in order to improve development times to rapidly meet new carbon streams and processing constraints for potential biorefinery configurations.

### SUMMARY OF THE INVENTION

The present invention provides for a method for increasing the production of a short chain alcohol, such as isoprenol or a diol, comprising: (a) providing a microbial strain comprising a genetic circuit comprising a short chain alcohol biosensor which allows growth of the microbial strain only when the microbial strain produces the short chain alcohol is produced, (b) screening for a cell with increased short chain alcohol production, and (c) optionally culturing or growing the cell to produce the short chain alcohol.

The present invention provides for a method for increasing the production of isoprenol or a diol, comprising: (a) providing a microbial strain comprising a genetic circuit comprising an isoprenol or diol biosensor which allows growth of the microbial strain only when the microbial strain produces isoprenol or the diol is produced, (b) screening for a cell with increased isoprenol or diol production, and (c) optionally culturing or growing the cell to produce isoprenol or the diol.

The present invention provides for a method for increasing the production of isoprenol, comprising: (a) providing a microbial strain comprising a genetic circuit comprising an isoprenol biosensor which allows growth of the microbial strain only when the microbial strain produces isoprenol is produced, (b) screening for a cell with increased isoprenol production, and (c) optionally culturing or growing the cell to produce isoprenol.

In some embodiments, the short chain alcohol is any alcohol with 2, 3, 4, 5, 6, 7, 8, 9, or 10 total carbon atoms, or a range of any two preceding values thereof. In some embodiments, the short chain alcohol comprises one, two, three, four, or five hydroxyl functional groups. In some embodiments, the short chain alcohol has one or two hydroxyl functional groups. In some embodiments, the short chain alcohol has one, two, or three C—C double bonds. In some embodiments, the short chain alcohol is isoprenol, or a diol. In some embodiments, the diol has 3, 4, or 5 carbon atoms, such as 1,2-propanediol or 1,5-pentanediol.

In some embodiments, the isoprenol biosensor is YiaYZ, or any isoprenol biosensor described herein, or a homologous variant thereof. In some embodiments, the cell with improved isoprenol productivity has about 2-, 3-, 4-, 5-, or 6-fold increased isoprenol production compared to the microbial strain prior to screening.

In some embodiments, the culturing or growing step (c) comprises the host cell growing by respiratory cell growth. In some embodiments, the culturing or growing step (c) takes place in a batch process or a fed-batch process, such as a high-gravity fed-batch process. In some embodiments, the culture comprises a biomass, such as a lignocellulosic biomass, or hydrolysate thereof. In some embodiments, the biomass is obtained from softwood feedstock (such as poplar), hardwood feedstock, grass feedstock, and/or agricultural feedstock, or mixture thereof.

In some embodiments, the culture or medium comprises a rich medium, such as LB (Lysogeny-Broth) or comprising one or more ingredients of LB, such as tryptone and/or yeast extract. In some embodiments, the culture or medium comprises hydrolysates derived or obtained from a biomass, such as a lignocellulosic biomass. In some embodiments, the culture or medium comprises one or more carbon sources, such as a sugar, such as glucose or galactose, or glycerol, or a mixture thereof. In some embodiments, the carbon source is fermentable. In some embodiments, the carbon source is non-fermentable. In some embodiments, the culture or medium comprises urea as a nitrogen source. In some embodiments, the culture or medium comprises an ionic liquid (IL).

In some embodiments, the invention comprises the use of a heterologous codon-optimized version of each nucleic acid encoding the described biosensor, which are optimized to the genetically modified host cell.

A genetic circuit was constructed tied intracellular biofuel concentration to cellular growth. This circuit was introduced into a strain of Pseudomonas putida that also produced the same jet-fuel precursor, isoprenol. A 12,000 member gRNA library targeting every gene in the P. putida genome was introduced into the strain. Since the genetic circuit allows growth only when the biofuel is produced, we screened for enriched gRNAs as a proxy for cells with improved isoprenol productivity. Preliminary screening has identified new strains that increase titers from 75 mg/L to 480 mg/L—a six-fold improvement. Moreover, this unbiased screening method has identified unintuitive/unexpected determinants of isoprenol titer.

Isoprenol is a volatile and colorless analyte that generally requires gas chromatography to quantitate. Methods enabling high throughput quantification would enable facile design/build/test/learn cycles necessary to increase strain productivity to economically viable levels. Here we leverage an isoprenol-responsive DNA element to build a biosensor-guided strain engineering pipeline. Instead of gas chromatography, we force the strain to produce isoprenol in order to grow. The DNA element of the biosensor was refactored to activate expression of an essential gene, pyrF. Since strain fitness was now tied to isoprenol production, we transformed in a 12,000 member gRNA library to query how genetic perturbations could increase growth as a simple screening methodology. Clones with better growth (and in turn more isoprenol production) were enriched in gRNA abundance, which were identified by nanopore whole genome sequencing. The corresponding deletion strains were generated and the improvement in isoprenol titer was confirmed by GC. The invention encloses both the methodology of a growth-enriching genetic circuit in conjunction with a gRNA library as well as the specific mutants which are determinants of isoprenol productivity.

The present invention also provides for the heterologous use of a DNA Sequence for dosage-dependent isoprenol quantification, such as a P. putida DNA Sequence for dosage-dependent isoprenol quantification. In some embodiments, the invention comprises: usi of a DNAng promoter sequence operably linked to a heterologous sequence encoding a fluorescent protein, such as mCherry, for the specific detection of an alcohol, such as a short chain alcohol, such as isoprenol. Such an synthetic construct is referred to as an alcohol biosensor, such as an isoprenol biosensor, which senses isoprenol. In some embodiments, a 254 nucleotide sequence immediately upstream of the start codon for the PP_2675 gene is placed in front of a ribosome binding site (RBS), such as an RBS+8 amino acid leader sequence followed by the mCherry coding sequence. When this plasmid comprising this construct is transformed into Pseudomonas putida KT2440, a dosage dependent mCherry fluorescent signal is observe that correlates with the concentration of initial exogenous isoprenol added to minimal salt growth media after a 24 hour incubation. The promoter sequence from PP_2675 is required for this isoprenol-dependent response; other promoters such as the sequence immediately preceding the sequencing encoding the PP_2682 gene fail to give similar activation. In P. putida, it is determined that the histidine kinase PP_2683 and the response regulator PP_2665 are required for this heterologous DNA sequence to respond linearly to exogenous isoprenol. In some embodiments, the gene(s) encode minimal protein component(s) needed to express and utilize the DNA-based biosensor in an organism other than P. putida.

Unlike some other microorganisms, Pseudomonas putida is natively able to consume isoprenol, a heterologously produced alcohol. Using RB-TnSeq data, the catabolic pathways that are responsible for consuming isoprenol are identified (M. G. Thompson et al., “Fatty Acid and Alcohol Metabolism in Pseudomonas putida: Functional Analysis Using Random Barcode Transposon Sequencing,” Appl. Environ. Microbiol. Vol. 86, No. 21, 2020; doi.org/10.1128/AEM.01665-20). These catabolic pathways are surmised to be transcriptionally controlled and hypothesized that promoter sequences driving expression of these catabolic pathways could be repurposed as an isoprenol-responsive elements. Effort has been put to identifying the upstream sensory machinery that would bind to the selected DNA promoter sequence, which has resulted in the generation of deletion strains and in turn complementation assays with the same genes reintroduced on plasmid-borne systems. In some embodiments, the isoprenol sensed is exogenous isoprenol. Exogenous isoprenol has been used to demonstrate the linearity of response. In some embodiments, the method can be used to enable high throughput screening methodologies since sample preparation is much faster/less laborious than with gas chromatography.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1: Functional Genomics Informs Biosensor Prospecting.

FIG. 2: Signaling Cascade Components Necessary for Alcohol Sensing in P. putida.

FIG. 3: Proposed Model of Isoprenol-YiaYZ Signaling Cascade.

FIG. 4: WT and ΔPP_2664 strains show differential response to short chain alcohol biosensor activation.

FIG. 5: the isoprenol biosensor as a proxy for heterologous isoprenol production in P. putida. The biosensor can also be used for a bioconversion pathway, not just to detect exogenous isoprenol.

FIG. 6: A Refactored Biosensor Links Growth to Improved Isoprenol Production.

FIG. 7: Survey of Heterologous Isoprenol Production Space via Parallelized Strain Engineering.

FIG. 8: mCherry induction using various compounds.

FIG. 9: Re-Analysis of RB-TnSeq Data Identifies Potential Signaling Cascades Amenable for Refactoring into Biosensors.

FIG. 10: Integrating the Biosensor Into Cellular Metabolism Enables High Throughput Screening Approaches.

FIG. 11: Experiment to determine the signal/noise ratio for different isoprenol responsive promoters.

FIG. 12: Two potential DNA promoter sequences were identified: PP_2675 and pp 2805.

FIG. 13: Short-term treatment of P. putida with exogenous isopentenol induces 100× expression of PP_2805.

FIG. 14: Three promoter-mCherry constructs are generated, but only pTE518 using a PP_2675 promoter showed a response to exogenous isopentenol.

FIG. 15: pPP_2675 likely responds to alcohols of varied chain lengths but not other common overflow metabolites.

FIG. 16: The PP_2675 promoter shows a dose-dependent response to exogenous isoprenol.

FIG. 17: Integrating the IP Biosensor Both Reduces Baseline mCherry Fluorescence and Increases Dynamic Response Range (bottom left: KT ΔPP_2675 pTE518; bottom right: KT ΔPP_2675 pTE520int, 4 indp. clones).

FIG. 18: KT2440 ΔPP_2675 has comparable isopentenol induction to WT, but the triple mutant (ΔPP_2675 ΔPP_3839 ΔPP_4064-4067) shows constitutive activation.

FIG. 19: PP_2675p represses its own baseline expression; PP_3839p enhances PP_2675 induction.

FIG. 20: A proposed HK/RR Signaling Cascade Pathway to express PP_2675.

FIG. 21: Effect of ΔPP_2683 and ΔPP2665 on the induction on PP_2675.

FIG. 22: 1,2-propanediol or 1,5-pentanediol also induce mCherry expression (top). KT2440 pTE518 is also able to demonstrate an over 20-fold induction by 1,2-propanediol (bottom).

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:
The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:
The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a value including 10% more than the stated value and 10% less than the stated value.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The terms “host cell” and “host microorganism” are used interchangeably herein to refer to a living biological cell, such as a microbe, that can be transformed via insertion of an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

The term “heterologous” as used herein refers to a material, or nucleotide or amino acid sequence, that is found in or is linked to another material, or nucleotide or amino acid sequence, wherein the materials, or nucleotide or amino acid sequences, are foreign to each other (i.e., not found or linked together in nature).

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Particular expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

The term “promoter,” as used herein, refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon (i.e., including the 5′ untranslated region of the mRNA, typically comprising 100-200 bp). Most often the core promoter sequences lie within 1-2 kb of the translation start site, more often within 1 kbp and often within 500 bp of the translation start site. By convention, the promoter sequence is usually provided as the sequence on the coding strand of the gene it controls. In the context of this application, a promoter is typically referred to by the name of the gene for which it naturally regulates expression. A promoter used in an expression construct of the invention is referred to by the name of the gene. Reference to a promoter by name includes a wildtype, native promoter as well as variants of the promoter that retain the ability to induce expression. Reference to a promoter by name is not restricted to a particular species, but also encompasses a promoter from a corresponding gene in other species.

A polynucleotide is “heterologous” to a host cell or a second polynucleotide sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

The term “operatively linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

### Enzymes, and Nucleic Acids Encoding Thereof

A homologous biosensor is an biosensor that has a polypeptide sequence that is at least 70%, 75%, 80%, 85%, 90%, 95% or 99% identical to any one of the biosensor described in this specification or in an incorporated reference. The homologous biosensor comprises or retains amino acid residues that are recognized as conserved for the biosensor. The homologous biosensor may have non-conserved amino acid residues replaced or found to be of a different amino acid, or amino acid(s) inserted or deleted, but which does not affect or has insignificant effect on the enzymatic activity of the homologous biosensor. The homologous biosensor has a biosensor activity that is identical or essentially identical to the biosensor activity any one of the biosensor s described in this specification or in an incorporated reference. The homologous biosensor may be found in nature or be an engineered mutant thereof.

The nucleic acid constructs of the present invention comprise nucleic acid sequences encoding one or more of the subject enzymes. The nucleic acid of the subject enzymes are operably linked to promoters and optionally control sequences such that the subject biosensor s are expressed in a host cell cultured under suitable conditions. The promoters and control sequences are specific for each host cell species. In some embodiments, expression vectors comprise the nucleic acid constructs. Methods for designing and making nucleic acid constructs and expression vectors are well known to those skilled in the art.

Sequences of nucleic acids encoding the subject biosensor s are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteuci et al. (1980) Tet. Lett. 521:719; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions (PCR; e.g., U.S. Pat. No. 4,683,195).

Each nucleic acid sequence encoding the desired subject biosensor can be incorporated into an expression vector. Incorporation of the individual nucleic acid sequences may be accomplished through known methods that include, for example, the use of restriction enzymes (such as BamHI, EcoRI, HhaI, Xhol, XmaI, and so forth) to cleave specific sites in the expression vector, e.g., plasmid. The restriction enzyme produces single stranded ends that may be annealed to a nucleic acid sequence having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired nucleic acid sequence are often cleaved with the same restriction enzyme, thereby assuring that the ends of the expression vector and the ends of the nucleic acid sequence are complementary to each other. In addition, DNA linkers may be used to facilitate linking of nucleic acids sequences into an expression vector.

A series of individual nucleic acid sequences can also be combined by utilizing methods that are known to those having ordinary skill in the art (e.g., U.S. Pat. No. 4,683,195).

For example, each of the desired nucleic acid sequences can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other Extension of this overlap by DNA polymerase produces a molecule in which the original sequences are “spliced” together. In this way, a series of individual nucleic acid sequences may be “spliced” together and subsequently transduced into a host microorganism simultaneously. Thus, expression of each of the plurality of nucleic acid sequences is effected.

Individual nucleic acid sequences, or “spliced” nucleic acid sequences, are then incorporated into an expression vector. The invention is not limited with respect to the process by which the nucleic acid sequence is incorporated into the expression vector. Those of ordinary skill in the art are familiar with the necessary steps for incorporating a nucleic acid sequence into an expression vector. A typical expression vector contains the desired nucleic acid sequence preceded by one or more regulatory regions, along with a ribosome binding site, e.g., a nucleotide sequence that is 3-9 nucleotides in length and located 3-11 nucleotides upstream of the initiation codon in E. coli. See Shine et al. (1975) Nature 254:34 and Steitz, in Biological Regulation and Development: Gene Expression (ed. R. F. Goldberger), vol. 1, p. 349, 1979, Plenum Publishing, N.Y.

Regulatory regions include, for example, those regions that contain a promoter and an operator. A promoter is operably linked to the desired nucleic acid sequence, thereby initiating transcription of the nucleic acid sequence via an RNA polymerase enzyme. An operator is a sequence of nucleic acids adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. An example includes lactose promoters (LacI repressor protein changes conformation when contacted with lactose, thereby preventing the LacI repressor protein from binding to the operator). Another example is the tac promoter. (See deBoer et al. (1983) Proc. Natl. Acad. Sci. USA, 80:21-25.) As will be appreciated by those of ordinary skill in the art, these and other expression vectors may be used in the present invention, and the invention is not limited in this respect.

Although any suitable expression vector may be used to incorporate the desired sequences, readily available expression vectors include, without limitation: plasmids, such as pSC101, pBR322, pBBRIMCS-3, pUR, pEX, pMR100, pCR4, pBAD24, pUC19; bacteriophages, such as M13 phage and 2 phage. Of course, such expression vectors may only be suitable for particular host cells. One of ordinary skill in the art, however, can readily determine through routine experimentation whether any particular expression vector is suited for any given host cell. For example, the expression vector can be introduced into the host cell, which is then monitored for viability and expression of the sequences contained in the vector. In addition, reference may be made to the relevant texts and literature, which describe expression vectors and their suitability to any particular host cell.

The expression vectors of the invention must be introduced or transferred into the host cell. Such methods for transferring the expression vectors into host cells are well known to those of ordinary skill in the art. For example, one method for transforming E. coli with an expression vector involves a calcium chloride treatment wherein the expression vector is introduced via a calcium precipitate. Other salts, e.g., calcium phosphate, may also be used following a similar procedure. In addition, electroporation (i.e., the application of current to increase the permeability of cells to nucleic acid sequences) may be used to transfect the host microorganism. Also, microinjection of the nucleic acid sequencers) provides the ability to transfect host microorganisms. Other means, such as lipid complexes, liposomes, and dendrimers, may also be employed. Those of ordinary skill in the art can transfect a host cell with a desired sequence using these or other methods.

For identifying a transfected host cell, a variety of methods are available. For example, a culture of potentially transfected host cells may be separated, using a suitable dilution, into individual cells and thereafter individually grown and tested for expression of the desired nucleic acid sequence. In addition, when plasmids are used, an often-used practice involves the selection of cells based upon antimicrobial resistance that has been conferred by genes intentionally contained within the expression vector, such as the amp, gpt, neo, and hyg genes.

When the host cell is transformed with at least one expression vector. When only a single expression vector is used (without the addition of an intermediate), the vector will contain all of the nucleic acid sequences necessary.

Once the host cell has been transformed with the expression vector, the host cell is allowed to grow. For microbial hosts, this process entails culturing the cells in a suitable medium. It is important that the culture medium contain an excess carbon source, such as a sugar (e.g., glucose) when an intermediate is not introduced. In this way, cellular production of the lactam compound ensured. When added, any intermediate is present in an excess amount in the culture medium.

Any means for extracting or separating the lactam compound from the host cell may be used. For example, the host cell may be harvested and subjected to hypotonic conditions, thereby lysing the cells. The lysate may then be centrifuged and the supernatant subjected to high performance liquid chromatography (HPLC) or gas chromatography (GC).

### Host Cells

In some embodiments, the host cells are genetically modified in that heterologous nucleic acid have been introduced into the host cells, and as such the genetically modified host cells do not occur in nature. The suitable host cell is one capable of expressing a nucleic acid construct encoding one or more biosensor s described herein. The gene(s) encoding the biosensor(s) may be heterologous to the host cell or the gene may be native to the host cell but is operatively linked to a heterologous promoter and one or more control regions which result in a higher expression of the gene in the host cell.

Each introduced biosensor can be native or heterologous to the host cell. Where the biosensor is native to the host cell, the host cell is genetically modified to modulate expression of the biosensor. This modification can involve the modification of the chromosomal gene encoding the biosensor in the host cell or a nucleic acid construct encoding the gene of the biosensor is introduced into the host cell. One of the effects of the modification is the expression of the biosensor is modulated in the host cell, such as the increased expression of the biosensor in the host cell as compared to the expression of the biosensor in an unmodified host cell.

The genetically modified host cell can be any bacterial cell capable of production of the lactam compound of the present invention in accordance with the methods of the invention.

In some embodiments, the host cell is a prokaryotic cell, such as a bacterial cell. In some embodiments, the host cell is a bacterial cell selected from the Escherichia, Enterobacter, Azotobacter, Erwinia, Bacillus, Pseudomonas, Klebsiella, Proteus, Salmonella, Serratia, Shigella, Ralstonia, Rhizobia, or Vitreoscilla taxonomical class. Bacterial host cells suitable for the invention include, but are not limited to, Escherichia, Corynebacterium, Pseudomonas, Streptomyces, and Bacillus. In some embodiments, the Escherichia cell is an E. coli, E. albertii, E. fergusonii, E. hermannii, E. marmotae, or E. vulneris. In some embodiments, the Corynebacterium cell is Corynebacterium glutamicum, Corynebacterium kroppenstedtii, Corynebacterium alimapuense, Corynebacterium amycolatum, Corynebacterium diphtheriae, Corynebacterium efficiens, Corynebacterium jeikeium, Corynebacterium macginleyi, Corynebacterium matruchotii, Corynebacterium minutissimum, Corynebacterium renale, Corynebacterium striatum, Corynebacterium ulcerans, Corynebacterium urealyticum, or Corynebacterium uropygiale. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida. In some embodiments, the Streptomyces cell is a S. coelicolor, S. lividans, S. venezuelae, S. ambofaciens, S. avermitilis, S. albus, or S. scabies. In some embodiments, the Bacillus cell is a B. subtilis, B. megaterium, B. licheniformis, B. anthracis, B. amyloliquefaciens, B. pumilus, B. brevis, B. aminovorans, or B. fusiformis. In some embodiments the bacterial cell is a Gram-positive bacterium, such as a Streptomyces species, such as any Streptomyces species or strain taught herein. In some embodiments, the Pseudomonas cell is a Pseudomonas putida cell. In some embodiments, the Pseudomonas putida cell is a Pseudomonas putida KT2440 cell. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida cell.

The genetically modified host cell can be any yeast capable of production of the lactam compound in accordance with the methods of the invention.

In some embodiments, the host cell is a yeast. Yeast host cells suitable for the invention include, but are not limited to, Yarrowia, Candida, Bebaromyces, Saccharomyces, Schizosaccharomyces and Pichia cells. In one embodiment, Saccharomyces cerevisae is the host cell. In one embodiment, the yeast host cell is a species of Candida, including but not limited to C. tropicalis, C. maltosa, C. apicola, C. paratropicalis, C. albicans, C. cloacae, C. guillermondii, C. intermedia, C. lipolytica, C. panapsilosis and C. zeylenoides. In one embodiment, Candida tropicalis is the host cell.

In some embodiments, the yeast host cell is a non-oleaginous yeast. In some embodiments, the yeast host cell is a basidiomycete. In some embodiments, the yeast host cell is an oleaginous yeast. In some embodiments, the oleaginous yeast is a Rhodosporidium species. In some embodiments, the Rhodosporidium species is Rhodosporidium toruloides. In some embodiments, the Rhodosporidium toruloides is strain IFO 0880.

In some embodiments, the host cell comprises a nucleic acid encoding the one or more biosensor s operatively linked to a promoter capable of expressing the one or more biosensor s in the host cell. In some embodiments, the encoding of the one or more biosensor s to the nucleic acid is codon optimized to the host cell. In some embodiments, the nucleic acid is vector or replicon that can stably reside in the host cell. In some embodiments, the nucleic acid is stably integrated into one or more chromosomes of the host cell.

In some embodiments, the providing step (a) comprises introducing a nucleic acid encoding the one or more biosensor s operatively linked to a promoter capable of expressing the one or more biosensor s in the host cell into the host cell.

In some embodiments, the culturing or growing step (b) comprises the host cell growing by respiratory cell growth. In some embodiments, the culturing or growing step (b) takes place in a batch process or a fed-batch process, such as a high-gravity fed-batch process. In some embodiments, the culture or medium comprises hydrolysates derived or obtained from a biomass, such as a lignocellulosic biomass. In some embodiments, the culture or medium comprises one or more carbon sources, such as a sugar, such as glucose or galactose, or glycerol, or a mixture thereof. In some embodiments, the carbon source is fermentable. In some embodiments, the carbon source is non-fermentable.

The present invention provides for a method for constructing a genetically modified host cell of the present invention, comprising (a) introducing a nucleic acid encoding the one or more biosensors operatively linked to a promoter capable of expressing the one or more biosensors in the host cell into the host cell.

One can modify the expression of a gene encoding any of the biosensors taught herein by a variety of methods in accordance with the methods of the invention. Those skilled in the art would recognize that increasing gene copy number, ribosome binding site strength, promoter strength, and various transcriptional regulators can be employed to alter an biosensor expression level.

Other objects, features, and advantages of the present invention will be apparent to one of skill in the art from the following detailed description and figures.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### An Isoprenol Biosensor for Combinatorial High Throughput Strain Engineering in Pseudomonas putida

Even as high throughput strain engineering methods have facilitated the discovery and optimization of new molecules, biofuel molecules remain particularly challenging. Unlike dyes, plastics, or food additives, alcohols have physical properties that impede development cycles from both analytical and biological standpoints. Here we report the development and characterization of an isoprenol biosensor in Pseudomonas putida KT2440, mediated by a native histidine kinase signaling cascade responsive to short chain alcohols. This biosensor provides rapid quantification of molecules including the bulk commodity polymer 1,2-propanediol as well as the bio-jetfuel precursor isoprenol (3-methyl-3-buten-1-ol). After delineating the core components of this transcriptional response we refactor the biosensor to identify competing bottleneck processes that limit heterologous isoprenol production in conjunction with a pooled Cpf1/Cas12a gRNA library targeting nearly all ORFs in the genome. In turn, this growth selection assay guided a combinatorial analysis of candidate genes for deletion (approximately ~59 to date) derived from this gRNA screen, increasing isoprenol titers from below the detection limit to approaching 900 mg/L in minimal salt media, incorporating existing rational engineering optimizations with discoveries from the biosensor guided analysis. Our method is generalizable to any circuit-based biosensor for potentially many other analytically challenging molecules.

Our group has evaluated the productivity of the alcohol isoprenol (3-methyl-3-buten-1-ol), platform commodity chemical with attractive properties specifically as a diesel fuel blendstock and precursor for the bio-jetfuel DMCO, across a number of microbial hosts. While there are strengths and drawbacks to producing isoprenol in any one microbe, Pseudomonas putida KT2440 has a naturally versatile catabolic profile competent of consuming isoprenol. The pathway for this catabolism was revealed using functional genomics data (RB-TnSeq-Thompson et al) and we speculated that this data could also provide a path to developing an isoprenol biosensor. Refactoring knowledge of the isoprenol catabolism signaling system could provide a readout for intracellular isoprenol, in turn enabling an alternative approach to strain engineering that did not rely on a mechanistic understanding of how a heterologous pathway modulates native metabolism.

We define a biosensor as the system where a ligand is recognized by a transcriptional activator, which in turn binds to a cognate DNA sequence driving transcription of a downstream reporter gene. The resulting gene expression linearly increases in response to the initial ligand concentration. There are undoubtedly other biosensor modalities (such as directly converting the ligand into a colored molecule) but the advantage of a biosensor that activates a genetic circuit is that the response can also be integrated back into cellular physiology for high throughput selection with growth based assay. For example, strains can be devised where cell growth is concomitant with the increased production of the ligand. Prokaryotic systems are also advantaged over eukaryotes for biosensor development as the determinants of ribosome translation efficiency are dictated by simple RBS sequences adjacent to the start codon as opposed to complex transcriptional regulators, enabling fine protein level expression tuning while maintaining inducibility.

While crystal structures exist for several catabolic enzymes in complex with short chain alcohols, few crystal structures are available to examine specific interactions between alcohols and regulatory proteins which could be repurposed for inducible gene expression. The BmoR transcriptional regulator from Thauera butanovorans has been reported to recognize the short chain alcohol n-butanol; more recently, Bahls et. al. developed an isopentanol biosensor by mutagenizing AlkS from P. putida GP01 for use in E. coli. AlkS is thought to natively recognize middle-chain n-alkanes as a ligand; by mutagenizing AlkS with error-prone PCR they broadened its substrate specificity to accept shorter chain alcohols as activating ligands (including isoprenol) and used this biosensor to demonstrate improvements of isopentanol titers up to 1.26 g/L in rich, undefined media. While this titer is high, rational engineering approaches have already demonstrated isopentanol titers exceeding 8 g/L in batch mode cultures, leaving open the question if biosensor derived findings can be synergistic or additive with other approaches. A biosensor specifically for isoprenol is necessary since it is the key precursor to di-methyl-cyclooctane (DMCO), an established sustainable aviation fuel. Isopentanol cannot be used in this process as it lacks the unsaturated double bond required to generate DMCO.

Here we show the development and characterization of an isoprenol biosensor, mediated by a native histidine kinase signaling cascade responsive to short chain alcohols including isoprenol and diols, such as the bulk commodity polymer 1,2-propanediol. After delineating its core components we refactor the biosensor to identify competing bottleneck processes that limit heterologous isoprenol production in conjunction with a pooled Cpf1/Cas12a gRNA library targeting nearly all ORFs in the genome. This growth selection assay in turn guided a combinatorial analysis of deletion mutants (>40 unique deletions) derived from this gRNA screen, increasing isoprenol titers from below the detection limit to over 750 mg/L in minimal salt media, incorporating existing rational engineering optimizations with discoveries from the biosensor guided analysis. Our method is generalizable to other transcriptional circuit-based biosensor that are impeded by analytical and biological challenges.

### Results

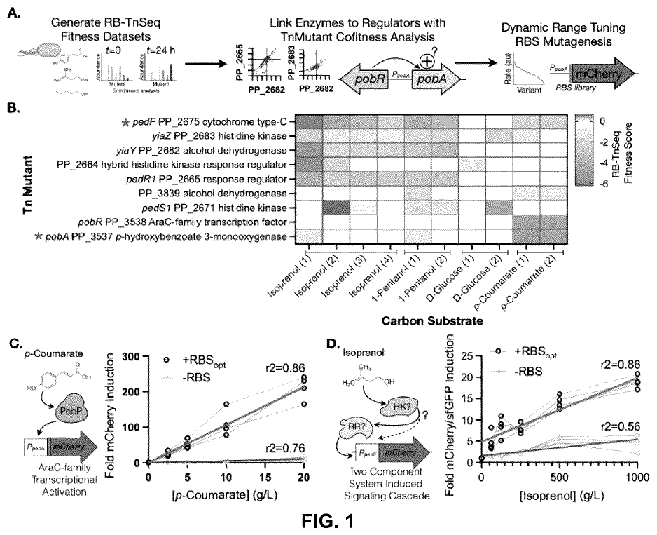

FIG. 1: Functional Genomics Informs Biosensor Prospecting.

### Development of a Dose Dependent Isoprenol Biosensor.

We developed a workflow to identify cis DNA sequences and their cognate ligand-DNA binding proteins for optimization as components of highly specific ligand-inducible systems monitored with a fluorescent readout (FIG. 1A). This pipeline relies on functional genomics data collected from diverse growth conditions with a 200,000-member P. putida KT2440 RB-TnSeq mutant library. Analysis of transposon-derived gene inactivations that have severe fitness defects under specified growth conditions could hint at endogenous inducible regulatory cascades, serving as the basis for inducible biosensors. The robustness of this method is derived from over 800 mutant co-fitness studies curated from diverse growth conditions accumulated from a decade of experimental research, all meeting stringent QC criteria. A co-fitness analysis is a linear regression for all fitness values for a given gene or condition. This correlation determines which other mutants have a similar trend among all tested conditions. Co-fitness values approaching 1 would indicate a perfect correlation between two transposon mutants. For this report, we first demonstrate the complete biosensor optimization workflow for an aromatic ligand and extend our reasoning to cover a more complex two component signaling system capable of sensing a diverse array of short chain alcohols and diols (FIG. 1).

Para-coumarate is a lignin derived aromatic that can be catabolized by P. putida but not other bioconversion hosts like E. coli. The concentration of this aromatic carbon source in various biomass pretreatment regimes is variable, and its concentration is quantified by laborious and delicate analytical methods like HPLC. A biosensor for p-CA would be of value to develop rapid workflows enabling high throughput characterization of biomass deconstruction methods. We evaluated two potential inducible signaling cascades that might be refactored into biosensors. p-CA is initially catabolized by PP_3356/Fcs to generate p-coumaryl-coA. To determine if the upstream DNA sequence PP_3356 could be used as a component of a ligand-inducible system, we asked if any transcriptional regulators were implicated in the cofit analysis. However, no transcriptional regulators had high cofitness values, implying these genes might be co-regulated by several transcriptional effectors. Based on this analysis the PP_3356 DNA promoter sequence was not an ideal candidate for a biosensor. In contrast, when we first filtered the RB-Tnseq database specifically for transcriptional regulators with low fitness values for p-CA, we identified PP_3538/pobR as a candidate ligand inducible transcription factor. PP_3538 was strongly cofit with an adjacent gene sharing the same upstream promoter sequence in a divergent promoter structure, PP_3537/pobA (p-hydroxybenzoate hydroxylase, cofit value ~0.81). pp 3537 is annotated as the fourth step in the p-CA degradation pathway, suggesting that pp 3538/PobR autoinduces bidirectional expression from its shared promoter sequence, inducing PP_3537 gene expression in the presence of a compatible ligand. This promoter sequence was cloned upstream of a mCherry sequence and tested for mCherry expression when p-CA was exogenously added to the media. Without tuning the RNA secondary structure of the PP_3537-mCherry construct, the initial mCherry fluorescence had low signal to noise, poor linearity, and was not reproducible across day to day replicates. Augmenting our promoter-mCherry design with an RBS was key to building a robust biosensor. We screened small, 40-member library of predicted candidate RBS variants. One RBS variant enabled a 100-fold increase in dynamic range and also improved the linear dynamic range for the analyte. This linear dynamic range in the 1 g/L concentration is well matched for current p-CA concentrations from lignocellulosic biomass. A simpler application of this workflow lacking the RBS optimization step on AraC-family transcriptional regulators that included a pobR-RFP biosensor was described in an earlier report. Crucially, by excluding the RBS optimization step, several potential ligand-responsive biosensors showed a limited or no predicted ligand dose response, indicating room for further analysis.

To generalize our extended biosensor development workflow we turned to isoprenol, a bioproduct rather than a carbon source. RB-TnSeq information has previously revealed that deletion of the cytochrome c oxidase, pedF (PP_2675), negatively impacts the ability of P. putida to grow on isoprenol as the sole carbon source (Thompson et al. 2020). As with the case for p-CA we searched for transcriptional regulators that might show a ligand inducible response in the presence of isoprenol with a cofitness analysis. We identified several promising regulatory elements that hinted at an inducible gene expression profile. Several histidine kinases were identified, including pedS2, yiaZ (PP_2683) and PP_2664 with co-fit values with pedF of 0.71, 0.62 and 0.47, respectively. Additionally, other genes that were found with similar co-fit values were the response regulators PP_2665 and pedR2 (co-fit values of 0.83 and 0.67, respectively) and the alcohol dehydrogenases yiaY (PP_2682), pedE and pedH (co-fit values of 0.80, 0.78 and 0.71, respectively). We investigated the fitness values of the new genes identified in the co-fitness analysis and found that both yiaY and yiaZ had a significant fitness defect on isoprenol (log 2 values of −2.2 and −1.2) highlighting their putative role in isoprenol degradation. In contrast to an intuitively obvious relationship between p-CA catabolism and upstream transcriptional regulators, activation of the isoprenol catabolic pathways was not as clear with several probable regulators acting at the same promoter element. Moreover histidine kinases are more complex two component signaling systems compared to single component signaling systems like PobR, and their genomic locations indicate dispersal across the genome.

Although we could not yet explain the ligand-induced signaling cascade, we tested if the upstream pedF DNA sequence was responsive to isoprenol as measured by production of a downstream fluorescent protein. (FIG. 1C). We selected a 254 bp non-coding DNA sequence located between genes pedF and pedE and we placed it upstream of a mCherry sequence. We used an existing RBS sequence optimized for mCherry described earlier. After transforming the P. putida wild-type strain with the synthetic P_{pedF}-RBS-mCherry plasmid construct, the strains were inoculated on M9 minimal media containing 2% of glucose and a range of isoprenol concentrations (0.62 to 2 g/L) determine the fold isoprenol response. As with the pobR biosensor, the lack of RBS optimization impaired biosensor activation, with poor dose-response, linearity, and reproducibility across biological replicates. The synthetic construct showed a 20× increase in signal over an uninduced control using 1 g/L exogenous isoprenol by 40 hours post exogenous isoprenol addition. Whereas the clone variability was initially high, we showed that the pedF promoter sequence is induced by isoprenol with a distinct linear dynamic range (FIG. 1C). As with the pobR-mCherry construct lacking an RBS, a similar pedF-GFP construct (previously discarded for poor performance during an earlier screening) showed similar poor inducibility and high day to day variability in experimental testing. We concluded the inducible mCherry fluorescence indicated the dose-dependent response of second ligand-responsive biosensor, demonstrating the generalizability of our method using functional genomics for the identification of biosensors at relevant substrate concentrations in applications including bioenergy research.

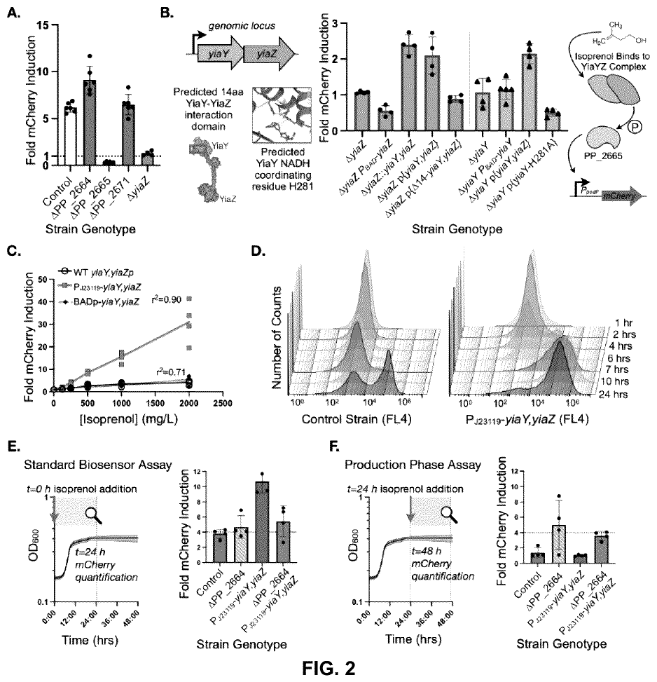

FIG. 2: Signaling Cascade Components Necessary for Alcohol Sensing in P. putida

### Biosensor-Driven Isoprenol Signaling Cascade Characterization

Our workflow to identify new ligands and their cognate activators was a path forward to understand the native regulatory mechanisms underlying isoprenol catabolism in P. putida. Since the biosensor responds to exogenous isoprenol, comparing biosensor fold activation between mutants would provide a quantitative and rapid assessment by revealing which components were required vs dispensable for pedF-RBS-mCherry activation. We used recombineering to make isogenic gene deletions of the histidine kinases and response regulators identified in the co-fit analysis in FIG. 1B. These knockout strains were transformed with the pedF-RBS-mCherry construct to examine differences in the activation of the isoprenol biosensor (FIG. 2A). First, we observed that deletion of PP_2665 ablated the biosensor response, indicating this was likely the primary response regulator involved in the isoprenol signaling cascade. Of the three most promising candidate histidine kinases, PP_2671 (pedS2), PP_2664 and PP_2683 (YiaZ), only the ΔyiaZ mutant strain showed loss of the biosensor activation, elucidating its required role in the signaling cascade. Intriguingly, deletion of PP_2664 slightly increased the biosensor fold response compared to a WT control (FIG. 2A), denoting a possible interaction that attenuates the response to isoprenol. PP_2664 encodes both a histidine kinase and response regulator domain and we hypothetized that it may be sufficient to activate a transcriptional cascade independent of a second response regulator. We concluded that the minimal isoprenol signaling cascade that activates the biosensor is mediated by the histidine kinase YiaZ that activates the PP_2665 response regulator by phosphorylation, which in turn binds and drives gene expression at the pedF promoter sequence.

A necessary component to mutant analysis is gene complementation to ensure genetic linkage to the observed phenotype. Reintroducing PP_2665 in the ΔPP_2665 strain under an inducible BAD promoter with ecotopic plasmid expression resulted in a constitutive mCherry fluorescence (Supplemental Figure AZ). This result indicated the construct's sensitivity to PP_2665 gene expression levels which impaired the dynamic range of the system but ultimately confirmed PP_2665 as a key component in the signaling cascade. In contrast, transforming the ΔyiaZ strain with a plasmid expressing yiaZ did not restore biosensor activity, indicating a failure to complement (FIG. 2B). Other yiaZ expression constructs were built using the native yiaYZ promoter and a constitutive PJ23119 Anderson promoter, but they also failed to complement biosensor activity and proteomics analysis confirmed that YiaZ was expressed at comparable levels to WT (FIG. 2B and Supplemental Figure AZ). The failure to complement the yiaZ deletion with ectopic reintroduction was unexpected and, since yiaZ is encoded in an overlapping frame with the alcohol dehydrogenase, yiaY, we hypothesized that a truncated gene sequence of yiaZ could have been subcloned if the annotated start codon was incorrect, likely resulting in the expression of a nonfunctional protein. To address this concern, we next tested the complementation of ΔyiaZ with a larger genomic 1.5 kb upstream sequence including yiaY to eliminate uncertainty regarding the YiaZ coding sequence. This sequence includes PP_2681, yiaZ and yiaY in a new plasmid construct and here we observed complementation ΔyiaZ, indicating that the larger sequence was sufficient to activate the biosensor (FIG. 2B). Furthermore, removing the yiaY start codon from the 3 kb PP_2681-yiaZ-yiaY sequence failed to complement, indicating the yiaY and yiaZ gene products were both required for biosensor activity and ruling out any contribution from PP_2681. Since there is an existing genomic copy of yiaY in the ΔyiaZ strain, we hypothetized that stoichiometric expression levels of yiaY and yiaZ are important for the biosensor activation.

To further investigate the role of the alcohol dehydrogenase in the signaling cascade, we generated a yiaY knockout strain and observed similar complementation requirements. While the ectopically plasmid expression of yiaY failed to restore the biosensor activity, the reintroduction of both yiaY and yiaZ complemented the loss of response (FIG. 2B). PFAM domain analysis revealed the YiaY protein contains a Fe+ alcohol dehydrogenase domain and sequence analysis with a ML-based contrastive model (CLEAN) suggests it may also function as a propanediol oxidoreductase. YiaZ is 41% identical to an iron-dependent alcohol dehydrogenase from Zymomonas mobilis ZM4 (PDB: 3OWO) and, while the deposited crystal structure was crystallized with NAD+, it does not contain the alcohol ligand. Fe+ alcohol dehydrogenases typically catalyze the oxidation of short chain alcohols to aldehydes (e.g. ethanol to acetaldehyde or butanol to butyraldehyde), but the specific analyte generated by YiaY from isoprenol and the role of this enzyme in the signaling cascade is unknown. Alcohol dehydrogenases are commonly multimeric homocomplexes that participate in complex multi-layer regulatory functions that include NAD+/NADH cycling in redox balance maintenance, alcohol/aldehyde detoxification in oxidative stress response, biofilm formation, alcohol-induced quorum sensing and gene expression modulation, but they are not known to be constituents of two-component signaling systems.

We sought to understand the role of YiaY in the protein complex formation as well as how its enzymatic activity could play a role in the signaling cascade. Since the biosensor activation was only observed when yiaY and yiaZ were both genomically or ectopically expressed, we hypothesized a model in which both would be constituent subunits of a multimeric complex, requiring a specific stoichiometry for activation (FIG. 2B). Using alphafold multimer analysis we modeled the potential interaction domains between YiaY and YiaZ. The leading N-terminal 14 amino acids of YiaY were predicted to interact with the N terminus of YiaZ and truncating these residues from YiaY blocked biosensor activation (FIG. 2B). In addition, when YiaY and YiaZ are coexpressed in E. coli they co-sediment by gel filtration analysis, consistent with the formation of a protein complex via a direct physical interaction (Supplemental Figure Y′). We also used Alphafill to determine the potential alcohol dehydrogenase active site and identified a NAD+ cofactor binding site that would likely be adjacent to the isoprenol catalytic domain. We mutated one of the residues that could form a coordinating salt bridge with NAD+ in YiaY (H281A) and we noted no biosensor activation in this point mutant (FIG. 2B). Taken together, these results indicate that YiaY is an integral accessory factor in the YiaZ/U.S. Plant Pat. No. 2,665 two component signaling pathway, and that the potential YiaYZ complex formation as well as its catalytic activity is essential for isoprenol to activate the regulatory cascade (FIG. 2B).

Our expanded knowledge of the isoprenol signaling cascade allowed us to evaluate the existing system to determine where optimizations to the biosensor response could be extracted. With the native YiaYZ signaling cascade, detection of fluorescent signal required 36-48 hours of incubation for signal maturation and the variation between biological replicates was high (FIG. 1C, FIGS. 2A & 2C). We hypothetized that the constitutive expression of yiaYZ could lead to a more rapid isoprenol detection by priming the two key signaling components for activation. Using recombineering we introduced either the high activity J23119 anderson promoter or a minimal BAD promoter to replace the native genomic yiaYZ promoter. Without introducing araC into the same strain, the BAD promoter would lack its repressor leading to constitutive expression. By testing the biosensor activation under a range of isoprenol concentrations, we compared the linear dynamic range of these constructs to a WT control strain (FIG. 2C) and observed that the kinetics of detectable fluorescence via biosensor activation and the linear dynamic range improved in the PJ23119-yiaYZ promoter variant, whereas the BAD promoter variant had only a negligible refinement (FIG. 2C). Differential proteomics analysis of the PJ23119-yiaYZ promoter variant identified increased cellular protein expression at multiple gene operons, suggesting the YiaYZ signaling cascade activated a diverse range of processes including many ped-family operons (i.e. PP_2663-PP_2680 & PP_4064-PP_4067); glucose uptake proteins and an uncharacterized transporter (KguDTKE and PP_3210); and PQQ synthase proteins (PP_0375-PP_0379).

Using flow cytometry to monitor the biosensor activation revealed kinetic improvements in signal maturation. In the PJ23119-yiaYZ promoter variant strain a distinct bimodal population emerged approximately 4 hours post exogenous isoprenol addition with a 25% of the population expressing at least 10-fold more mCherry and, by 7 hours, nearly 80% of cells were mCherry positive (FIG. 2D). In contrast, only 20% of the WT control strain population was mCherry positive 24 hour post-isoprenol addition, and had a maximum of 5-fold mCherry induction (FIG. 2D). Overall, the synthetic constitutive Anderson promoter upstream of yiaYZ had successfully improved biosensor performance in important metrics including dynamic range, kinetics, and % CV between biological replicates.

To further characterize the ability of the synthetic biosensor to detect isoprenol we decided to investigate its activity under different strain metabolic conditions. Our conventional isoprenol assay uses log-phase cultures harboring the biosensor to detect exogenously added isoprenol at the start of the timecourse (FIG. 2E). This setup is relevant for assessing isoprenol present in environmental samples but may not recapitulate a microbial production scenario, where isoprenol predominantly accumulates during stationary phase after glucose has been completely consumed. We modified our standard biosensor induction assay by growing the biosensor-harboring strains on M9 media and adding isoprenol to the overnight saturated cultures (FIG. 2F). Interestingly, we observed that the 10-fold activation signal of the pJ23119-yiaYZ biosensor strain was lost when exogenous isoprenol was added to stationary phase cultures instead of log-phase cultures (FIG. 2E & FIG. 2F). The ideal biosensor is required to detect isoprenol regardless of the metabolic state of the strain harboring the biosensor, especially if the isoprenol biosensor was to be integrated with a heterologous isoprenol production pathway. To enable stationary phase detection of isoprenol, we revisited and applied our expanded knowledge of the biosensor signaling cascade. Previously we noted that deleting the histidine kinase pp 2664 resulted in strains that exhibited a higher mCherry fold induction (FIG. 2A), but increased variability over WT from day to day. We re-examined the deletion of pp 2664 under both biosensor induction assay conditions and showed that the biosensor activity was restored in stationary phase cultures in ΔPP_2664 strains (FIG. 2F). While histidine kinases have a canonical configuration of homodimers, it has been reported that they can also form heterodimers. We hypothesized that YiaZ/PP_2664 heterodimers could form but are less efficient in activating the signaling cascade. By eliminating the competition from PP_2664/YiaZ heterodimers forming in the cell and restricting it to YiaZ homodimers, the absolute mCherry signal observed is increased in log-phase cultures (FIGS. 2A & 2E) and restored in stationary-phase cultures (FIG. 2F). Both the ΔPP_2664 strain and the PJ23119-yiaYZ ΔPP_2664 strain were able to detect and respond to isoprenol in stationary phase but there was more variability in the biosensor response when yiaYZ was expressed under its native promoter. The constitutive 23119 promoter had less variability but the overall fold response was reduced compared to the standard assay, suggesting a stronger stationary phase promoter could yield further improvement. We concluded from this new assay that PP_2664 is a competitive biosensor inhibitor which can dampen the stationary phase response.

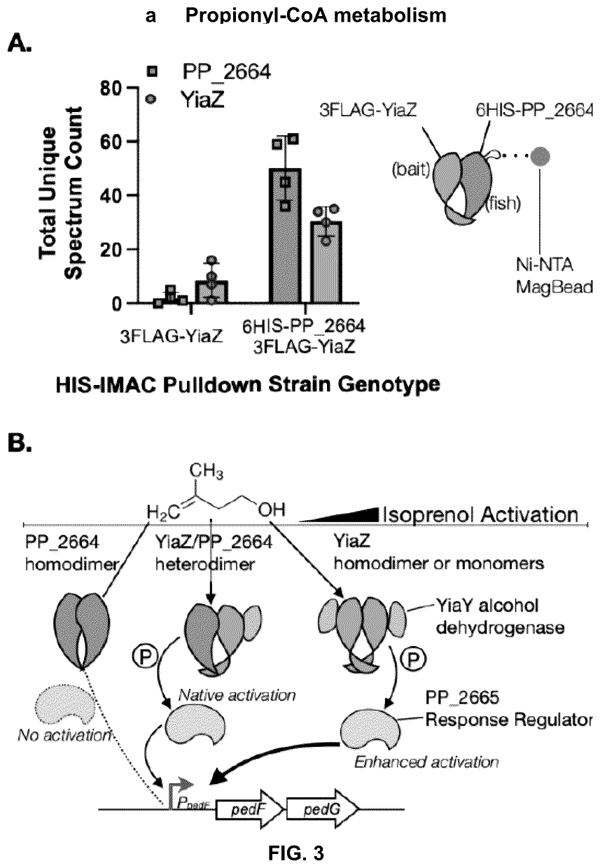

FIG. 3: Proposed Model of Isoprenol-YiaYZ Signaling Cascade.

The previous genetic analysis suggests that YiaZ and PP_2664 can reciprocally modulate their respective activities, but does not provide evidence for a physical interaction. We have not been able to obtain a full length protein crystal structure of YiaZ or PP_2664 to confirm homo/heterodimer formation by SAXS. Expressing YiaZ in E. coli does not appear to be stable without coexpression with YiaY, but the YiaZ PAS domain expressed and purified in E. coli eluted as a homodimer (supplemental figure XY), suggesting the full length YiaZ would be competent to behave similarly in P. putida. Alphafold multimer analysis also predicted that the full length YiaZ and PP_2664 proteins could form both homodimers and heterodimers along contacts between their coiled-coil domains.

We provide evidence of a physical interaction between YiaZ and PP_2664 using an affinity pulldown assay with an N-terminal in-frame 6HIS epitope tagged allele of PP_2664 expressed in P. putida (FIG. 3A); the gene encoding this protein was co-expressed on a plasmid along with an N-terminal in-frame 3FLAG epitope tagged allele of YiaZ. As a control, we also transformed P. putida with a plasmid expressing only the gene encoding 3FLAG-yiaZ. Both of these plasmids were induced and the lysed cell pellets were prepared for incubation with nickel coated magnetic beads to affinity purify 6HIS-PP_2664. After the beads were washed to remove nonspecific protein binding, the purified samples were analyzed by mass spectrometry. We determined that 3FLAG-YiaZ was enriched by 3.6 fold on nickel beads when 6HIS-U.S. Plant Pat. No. 2,664 was present in the sample, indicating that these two histidine kinases could stably interact consistent with the formation of a larger complex. This evidence of protein affinity between PP_2664 and YiaZ supports a potential PP_2664/YiaZ heterodimer as a functional unit in the cell that can exist alongside the PP_2664 and YiaZ homodimers.

Our genetic and biochemical experiments have shed insight into the mechanism underlying the isoprenol signaling cascade driving downstream expression of the larger transcriptional response induced by YiaZ and we synthesized our findings in FIG. 3B. In this model, P. putida expresses PP_2664 and YiaZ histidine kinases under the presence of compatible ligands, which are competent to form homo and heterodimers at an unknown dynamic rate. YiaY binds to YiaZ and might enzymatically reduce isoprenol to an aldehyde (i.e., 3-methyl-3-butenal) and help physically stabilize the modified ligand for recognition by YiaZ. In turn, activated YiaZ autophosphorylation or trans-phosphorylation would cause the subsequent phosphorylation of PP_2665, changing to its active confirmation and allowing it to bind to several genomic loci, facilitating the gene expression in conjunction with a sigma factor. The degree of isoprenol pathway activation in a population is variable as both the monomer protein expression level and ratio of homodimers to heterodimer can differ, both contributing to heterogeneity in response (FIG. 2D). Since we previously observed that the deletion of yiaZ abolishes the biosensor response (FIG. 2A) we concluded that the PP_2664 homodimer is not activated by isoprenol. In addition, deletion of PP_2664 noted that the yiaZ homodimer has a better response to isoprenol (FIG. 2A, 2E & 2F).

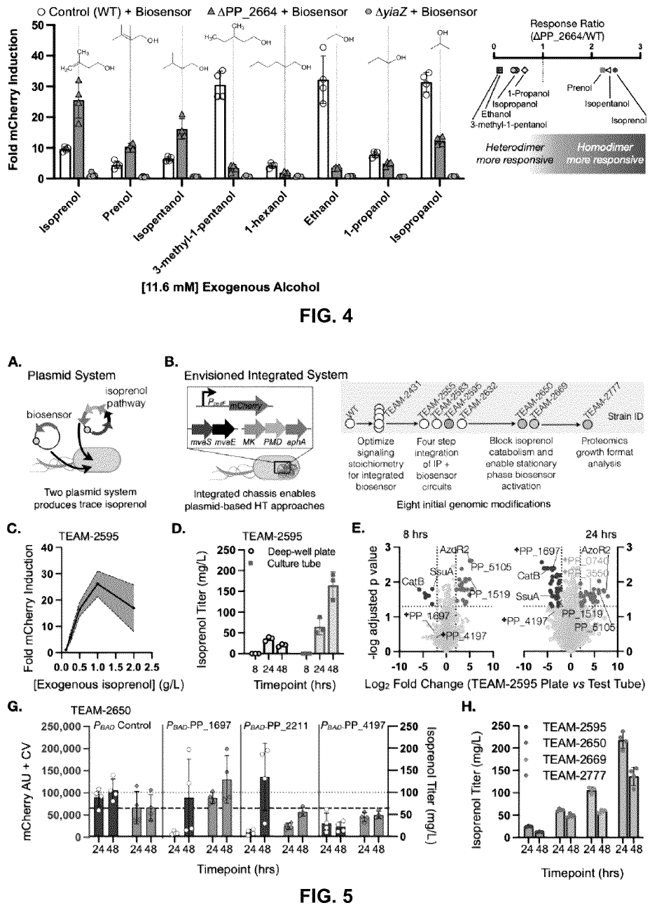

FIG. 4: WT and ΔPP_2664 strains show differential response to short chain alcohol biosensor activation.

### Alcohol Signaling Regulation is Tuned by Histidine Kinase Dimer Configuration

Having clarified how histidine kinase expression was important for the kinetics of signal transduction in the biosensor reporter assay, we next turned our attention to characterizing ligand specificity. While P. putida does not secrete ethanol or other alcohols as overflow metabolites, it was important to determine what other potential ligands would be compatible with the potential YiaZ active site. Under normal cultivation conditions P. putida cellular metabolites did not substantially contribute to the basal biosensor activation as the mCherry signal of strains transformed with the biosensor plasmid had similar background fluorescence levels to un-transformation strains. However, if isoprenol was produced in other microbial hosts like E. coli, other metabolic byproducts such as acetate and ethanol could interfere with the biosensor response. We repeated the biosensor induction assay using alternative metabolites and we determined that, while ethanol was also competent to induce the biosensor, amino acids and carbon sources including lysine, succinate, valine, and glycerol showed no biosensor activation (Supplemental Figure YA). Additionally, based on YiaZ's CLEAN annotation as a propanediol oxidoreductase, we confirmed that the biosensor can also be activated by 1,2-propanediol and 1,5-pentanediol (Supplemental Figure YB).

We extended our ligand specificity analysis to a broader range of short chain alcohols in WT, ΔyiaZ and ΔPP_2664 strains to determine if alcohol activation was modulated by preferential formation of YiaZ or PP_2664 homodimers (FIG. 4). Using equimolar alcohol concentrations, we observed that branched chain alcohols with the branch in the 1 position (isoprenol and isopentanol) showed strong, 25-fold induction in the ΔPP_2664 strain and was approximately 2-fold stronger than in WT (FIG. 4). Branched molecules with the branch at the 2 position like 3-methyl-1-pentanol had the opposite preference, showing greater activation in WT over the ΔPP_2664 strain background. Linear alcohols like ethanol and hexanol had variable fold responses ranging from 4-fold up to 40-fold and were preferentially activated in the WT strain background. The branched 2 carbon alcohol, isopropanol, had similar fold activation levels like isoprenol, but with the opposite dimer preference where activation in WT was preferred. In all cases tested, the absence of YiaZ blocked downstream biosensor activation, indicating pp 2664 was not able to activate PP_2665 with other potential ligands, suggesting PP_2664 homodimers cannot drive transcription at the pedF locus. We include a summary of the WT to ΔPP_2664 response ratio as an inset panel in FIG. 4.

FIG. 5—the isoprenol biosensor as a proxy for heterologous isoprenol production in P. putida. The biosensor can also be used for a bioconversion pathway, not just to detect exogenous isoprenol.

Designing the biosensor allowed us to characterize the components of the isoprenol signaling cascade, the signaling regulation by the histidine kinase dimer formation and the specificity for several alcohols and diols. However, while the plasmid-borne isoprenol biosensor had clear applications in detecting alcohols present in environmental samples or microbial communities, we were primarily interested in revealing cellular determinants of heterologous isoprenol productivity in P. putida and using the biosensor as a method for increased heterologous isoprenol production was an obvious strategy. As commodity chemicals, the technoeconomic analysis of biofuel molecules is an important consideration in any bioconversion process and these same parameters were germane for where the biosensor should be deployed. For example, costly reagents like antibiotics, rich undefined media (yeast extract), and pathway inducers were to be avoided. These guidelines suggested we engineer a unified P. putida strain containing both, the biosensor and isoprenol production modules, integrated in the genome for potential real-time product formation monitoring.

While a two-plasmid strategy for biosensing and isoprenol production was the fastest to prototype in P. putida, the resulting strain was slow growing and did not produce detectable isoprenol. As outlined in FIG. 5B, genomic integration of both modules was attempted with the following strain construction strategy. First we tested several synthetic promoters to constitutively express either yiaYZ and/or PP_2665 for increased biosensor sensitivity. The optimal design (a constitutive promoter for PP_2665) was then transformed with the isoprenol pathway split across two integration loci. The pathway was modified to replace the original arabinose inducible BAD promoter with a more economical crystal violet inducible promoter (citation). An additional copy of the biosensor was integrated at a second locus (increasing sensitivity) and finally, PP_2664 and PP_2675 were deleted to enable stationary phase biosensor function and to block isoprenol catabolism respectively. At several intermediate stages we assessed both isoprenol productivity and biosensor sensitivity.

The first viable strain design with both portions of the isoprenol pathway genomically integrated (TEAM-2595) displayed acceptable biosensor sensitivity. Its response to exogenous isoprenol was linear up to 500 mg/L and exhibited a maximum 30-fold signal increase over background.

Knowing that aeration differences from headspace can impact strain productivity, we assayed isoprenol titers in both the deep well and 5 mL culture tube formats. We observed that TEAM-2595 grown in 5 mL culture tubes produced up to 250 mg/L isoprenol, whereas the same strain grown in the deep well plate format produced approximately 30 mg/L. From this observation we hypothesized that cell physiology was favorable for isoprenol production due to aeration differences in the 5 mL tube format, which would be captured in a differential proteomics analysis. In turn, this information could be applied to overexpress or remove regulatory pathways limiting productivity in the deep well plate format. Samples were harvested from these two formats to compare differentially expressed proteins at log phase and stationary phase.

With a Bonferroni adjusted p-value, roughly 2% of detectable proteins were differentially expressed at the 8, 24, and 48 hour timepoints, with about twice as many differential expressed at the 24 hour timepoint (71 proteins) compared to the 8 hour timepoint (37 proteins). Several aromatic-related catabolism proteins (CatCBA-1) were upregulated in the test tube format, as well as several alcohol and aldehyde dehydrogenase proteins. A cold shock response protein was also upregulated. A small number were membrane transport proteins suggesting upregulation of sulfate and YYY nutrient transporters. The remaining proteins either had unknown function or finally, were the desired transcriptional regulators. PP_1697, pp 4197 & pp 4100 were uncharacterized GntR, GntR, and Cro/Cl-family regulators with no fitness defects by RB-TnSeq analysis. However, only PP_1697 and PP_4197 were overexpressed in the 5 mL format at all timepoints, and PP_4100 expression was not statistically significant at the 8 hour or 48 hour timepoints. We hypothesized that overexpressing PP_1697 or PP_4197 could further drive isoprenol titers in the deep well plate by forcing expression of a diverse range of gene targets, as opposed to overexpressing a single metabolic reaction. As a control we included a regulator of acyl-coA synthetase, the distal central metabolite precursor used to generate isoprenol.

To determine if the biosensor could be used as a proxy for isoprenol production in the unified biosensor-production strain, we transformed these overexpression constructs into TEAM-2650, a unified biosensor-production strain with the ΔPP_2664 deletion. Isoprenol production and plasmid expression were both induced at the start of the production time course and we monitored both mCherry and isoprenol kinetics at 24 and 48 hours post induction. The empty vector control displayed comparable mCherry signal (100,000 au) at both timepoints and approximately 55 mg/L isoprenol. Both PP_2211 and PP_1697 displayed increased mCherry signal (up to 200,000 au) at the 48 hour timepoint, but had reduced mCherry fluorescence at the 24 hour timepoint (approximately 10,000 au). The isoprenol titer in the P_{BAD}-PP_2211 strains however was a false positive as GC-FID analysis indicated titers were lower at the 24 hour timepoint, but did not exceed 55 mg/L in the 48 hour sample as expected from the mCherry fluorescence. It is possible that P_{BAD}-PP_2211 strains accumulate an isoprenol pathway intermediate that can also cross-activate the biosensor. On the other hand, the P_{BAD}-PP_1697 strains had fluorescence values that corroborated the isoprenol titer when verified by GC-FID; the 24 hour timepoint was an under-estimate from mCherry fluorescence with samples producing 87 mg/L, and the 48 hour timepoint producing an average of 129 mg/L with two replicates producing 150 mg/L and 200 mg/L isoprenol. These same two biological replicates were matched in generating high mCherry values. Finally, the P_{BAD}-PP_4197 showed reduced mCherry signal and isoprenol titer (30,000 au and 40 mg/L, respectively) hinting that PP_4197 overexpression was inhibitory towards isoprenol titers and conversely, implying this gene should be deleted to improve isoprenol titers. Since we observed high variability between biological replicates with the plasmid-borne overexpression construct, we used recombineering to directly introduce the synthetic constitutive Anderson promoter upstream of the PP_4197 start codon; this genomically integrated strain led to stable isoprenol titer improvements over its parental strain with titers approaching 250 mg/L in the deep well plate format (FIG. 5H). Overall the biosensor provided information on whether a strain should be down-selected for additional confirmation by GC, but could also generate false positive hits. These experiments highlight the complexity of heterologous product formation (and its metabolic intermediates) compared to detecting a fixed concentration of a purified analyte added to culture media at the start of the time course.

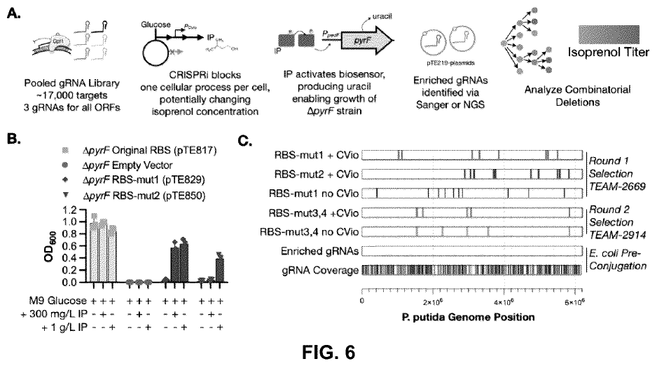

FIG. 6: A Refactored Biosensor Links Growth to Improved Isoprenol Production.

To link the biosensor to a selection regime rather than a screen-we deployed the biosensor as a genetic circuit embedded into cellular metabolism rather than as a simple endpoint readout for isoprenol concentration. In this configuration, higher concentrations of isoprenol were linked to cell growth under selective conditions, where the pedF promoter drove expression of an essential gene. Strains were interrogated with high throughput functional genomics methods to identify mutants that improved pedF activation as a proxy for higher isoprenol titers. We replaced the mCherry ORF downstream of pedF with pyrF. PyrF/PP_1815 is an orotidine 5′-phosphate decarboxylase required for uracil biosynthesis and its deletion leads to an uracil auxotrophy. If a ΔpyrF strain carries only a synthetic pedF-pyrF construct, it will be conditionally auxotrophic without isoprenol induction. Only when isoprenol is present above a certain threshold would pyrF expression be sufficient for cell growth. By transforming this strain with a plasmid-born Cpf1/gRNA CRISPRi library containing ~17,000 gRNAs targeting nearly all genes with 3 unique gRNAs, we interrogated the relationship between CRISPRi knockdown and the relationship with isoprenol titer. Clones that produced above an isoprenol threshold titer would grow, generating the selection needed to identify the rare winners from the larger pool of candidate gRNAs. As CRISPRi knockdowns may not be fully penetrant, the selection for improved growth eliminates background from nonfunctional gRNAs as they will not block RNA expression or increase isoprenol levels, eliminating them from analysis.

The key element required for implementation was the identification of a new RBS sequence downstream of the pedF promoter sequence that demonstrated a isoprenol-dose response matched to titers in the 500 mg/L range. Merely subcloning pyrF downstream of the existing pedF RBS sequence to replace mCherry was insufficient; the existing mCherry RBS constitutively complemented a ΔpyrF strain in minimal media. We mutagenized the pedF-pyrF RBS sequence and screened for new clones that demonstrated isoprenol responsive growth. The mutant plasmids contained additional modifications in the pyrF coding sequence in addition to the new RBS sequence suggesting additional mutations were necessary to reach the desired inducible range. The isoprenol-inducible growth phenotype was validated by re-transformation into both ΔpyrF and TEAM-2650 ΔpyrF strains; afterwards we used conjugation to introduce the pooled gRNA library as a second plasmid. Six replicate wells for each strain/plasmid combination were prepared for growth in minimal media with or without crystal violet to induce the pathway, and after 24 hours two of these replicates (with inducer) showed robust turbidity. RBSmut-1 had a lower isoprenol threshold for growth and there was faint growth without addition of the isoprenol pathway inducer. RBSmut-2 did not show growth without added inducer, consistent with its higher threshold for isoprenol. We collected biomass from several of these wells from both the control and with inducer samples, and enriched gRNAs were identified with nanopore transposase-based amplicon sequencing. The read depth of this method captures ~2,000 gRNAs per sample at a cost and speed superior to Sanger sequencing. While Illumina WGS is capable of capturing the entire gRNA library diversity with high fold coverage, both the turnaround time (2-3 weeks vs ~12 hours) and cost per run ($95 vs $15) were not competitive with the nanopore method. Moreover due to the nature of the selection method we had constructed, the additional information on library diversity would not be used for next steps in strain engineering.

We applied this selection regime in two sequential rounds using four pedF-RBS-pyrF variants and three different strains. The first round screened the gRNA library in a TEAM-2650 ΔpyrF variant where the isoprenol titer was ~50 mg/L. The enriched gRNAs were used to guide four rounds of combinatorial deletion and rational engineering to increase titers up to 700 mg/L. The two highest resulting strains were then made auxotrophic for uracil with a ΔpyrF mutation, new pedF-RBS-pyrF constructs were designed to exceed the higher baseline isoprenol titers generated in those new strains, and the pooled gRNA library was screened once more. Overall, 35 gene deletions were selected from these biosensor-linked selection assays for analysis and spanned functional categories including membrane efflux pumps, uncharacterized transcriptional regulators, unrelated metabolic reactions, or had unknown function. Coupled with our existing tools in rational strain engineering we describe the combinatorial search space below.

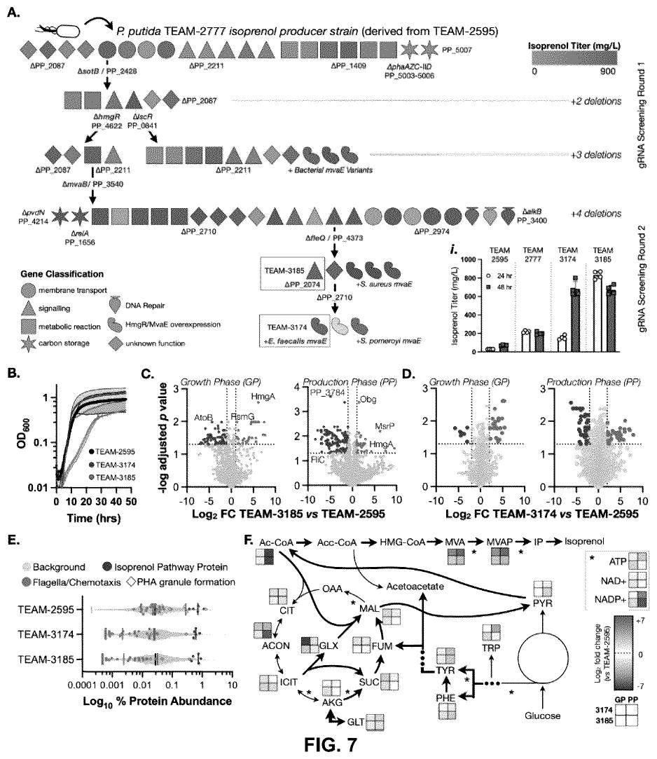

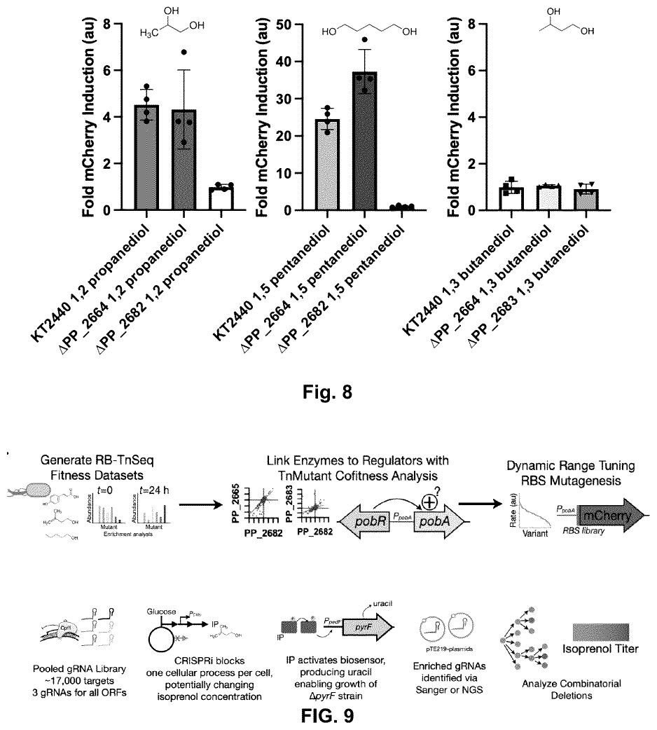

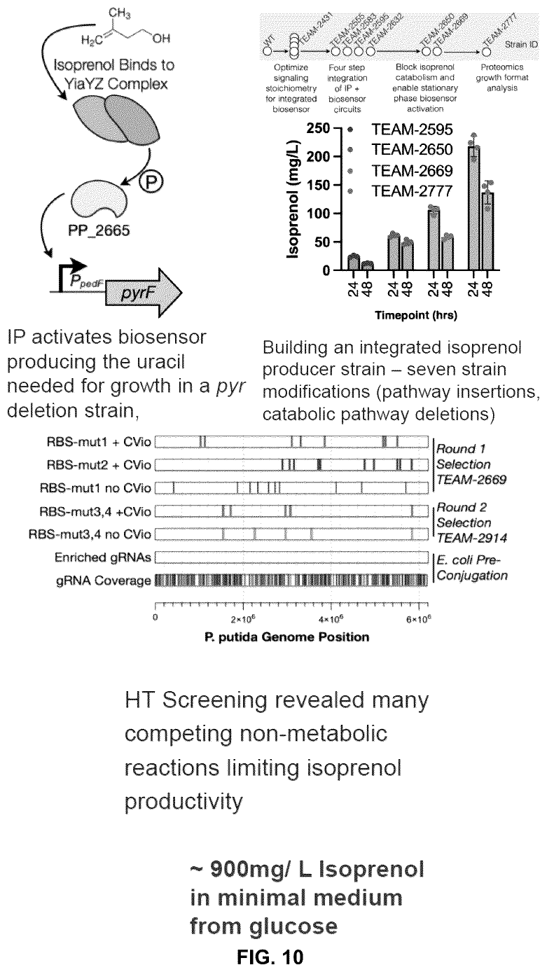

FIG. 7: Survey of Heterologous Isoprenol Production Space via Parallelized Strain Engineering.

### Data Availability

The Illumina paired end reads use for whole genome resequencing and mutation analysis have been deposited at NCBI under BioProject PRJNA1226229 and linked to BioSamples SAMN46924002 (Strain TEAM-2595), SAMN46924003 (Strain TEAM-3175), and SAMN46924004 (Strain TEAM-3185). Proteomics data are deposited at Panorama web.

### Methods

This medium was always used unless otherwise indicated. M9 minimal medium used here was modified from the “NREL” formulation 92 to 47.9 mM Na2HPO4, 22 mM KH2PO4, 8.56 mM NaCl, 2 mM MgSO4, 100 μM CaCl2 with 1× trace metals solution (Catalog Num. T1001, Teknova Inc, Hollister CA), 2% glucose, 70 mM (NH₄)₂SO₄ and 30 mM of 3-(N-Morpholino) propanesulfonic acid (MOPS; (Sigma Catalog Num. M1254)) and set to a pH of 7.0 was used to test the performance of the strains.

### Metabolomics

Organic acids were quantified by liquid chromatography and mass spectrometry (LC-MS) using the method described in Wang G, Kakumanu R, Amer B, Akyuz Turumtay E, Baidoo EEK (2025) Reversed phase LC-MS analysis of organic acids involved in the tricarboxylic acid cycle. Protocols.io. DOI: dx.doi.org/10.17504/protocols.io.bp216dz15vqe/v2. All other metabolites were quantified by LC-MS via a BEH Amide column (2.1 mm ID, 100 mm length, 1.7 μm stationary phase particle diameter; Waters Corporation, MA, USA) and the appropriate guard column (Waters Corporation, MA, USA) using the method described in Amer B, Kakumanu R, Baidoo EEK (2022) HILIC-MS analysis of central carbon metabolites in gram negative bacteria. Protocols.io. DOI: dx.doi.org/10.17504/protocols.io.4r312opzxv1y/v1. Metabolites were quantified via external calibration curves or chemical standards (for relative quantification). Data acquisition was performed via the Agilent MassHunter Workstation software (version 8). Data processing and analysis were performed via Agilent MassHunter Qualitative Analysis (version 6) and Profinder (version 8) software, respectively.

### Isoprenol Production Assays

The strains that were used to assay isoprenol production were struck to singles on LB agar plates from cryostorage and kept for no longer than two weeks. When new strains were tested, the control strains were also revived at the same time. LB cultures were prepared in test tubes from a single colony with a fill volume of 5 mL and incubated at 30° C. with 200 rpm orbital shaking and 40% humidity. Two adaptations on M9 minimal media were performed before starting the production run. In the first adaptation, 200 μL from the overnight LB cultures were inoculated in 5 mL of M9 minimal media in test tubes. The second adaptation was performed the next day using 24-deep well plates and inoculating 100 μL from the saturated culture in 1.5 mL fresh M9 medium. These plates were sealed with a gas-permeable film and incubated at 30° C. with 1,000 rpm linear shaking and 70% humidity. After 24 hours, the strains were ready to start the production run and OD₆₀₀ readings were taken to inoculate them with a starting OD₆₀₀ of 0.15. The production run was performed in 24-deep well plates using a fill volume of 1.5 mL and a gas-permeable film. The same M9 medium used during the adaptations was aliquoted and the isoprenol production inducer, crystal violet (CV, #ABC), was added to a final concentration of 1 μM from a stock solution of 2.5 mM before adding the medium into the deep-well plates. Several concentrations of CV were tested and 1 μM resulted in the best production in our initial strain. Tape was added to the plates on top of sealing film covering half of each well to reduce evaporation, but still allowing a proper air flow. All production runs were performed in quadruplicate and, when two different clones from the same strain were tested, two biological replicates were prepared from each strain. The M9 minimal medium was freshly prepared for each production run and discarded when precipitation occurred. 200 μL samples were collected after 24 h and 48 h and saved at −80 C if they were not extracted at the same moment. If overexpression plasmids were used, gentamycin was added to ensure the strains were maintaining the plasmids and arabinose was supplemented to a final concentration of 1% from a 20% stock solution in order to induce the gene expression unless otherwise indicated for basal induction.

### Isoprenol Extraction

Isoprenol quantification was performed using the gas chromatography-flame ionization detection (GC-FID, Thermo Focus GC) equipped with a DB-WAX column (15 m, 0.32 mm inner diameter, 0.25 μm film thickness, Agilent, USA). The GC oven was programmed as follows: 40° C.-100° C. at 15° C./min, 100° C.-230° C. at 40° C./min finally, held at 230° C. for 2 min. The inlet temperature was 200° C.

All samples were saved in Eppendorf tubes and stored at −80 C if they were not extracted and analyzed after being harvested. If frozen, samples were thawed at RT/4 C. Ethyl acetate (ABC) was used as the extraction solvent and it was prepared aliquoting 50 mL in a glass bottle and adding 100 mg/L of n-butanol (ABC) as the internal standard. To extract isoprenol, 200 μL of the prepared ethyl acetate was added to 200 μL culture samples and the mixture was vortexed at ABC rpm for 35 minutes in ABC. Next, samples were centrifuged 2 minutes at 15,000×g, and 100 μL were taken from the ethyl acetate phase and added to gas chromatography vials (ABC) containing inserts (ABC) and capped using ABC caps.

Isoprenol standards were always prepared diluting isoprenol (ABC) to a concentration of 2 g/L in a 25 mL sample of the same minimal media used for the production run. To account for isoprenol loss during extractions, a 200 μL aliquot of the medium containing 2 g/L of isoprenol was mixed with 200 μL of the same prepared ethyl acetate used to extract the culture samples. Isoprenol was extracted using the same protocol described above. Blanks were also prepared using the same ethyl acetate and ran after 30 samples to ensure the accuracy of our detections.

### Evaporation Correction

To assess the isoprenol evaporation loss during the cultivation at 30 C, M9 minimal media was added to deep-well plates containing a range of isoprenol concentrations from 2 g/L to 125 mg/L. These defined concentrations were done in triplicates and incubated simulating the production run conditions. To prepare these plates, 3 mL of M9 media containing 2 g/L of isoprenol was added to the first row of wells, and 1.5 mL of the same medium without isoprenol was added to the remaining wells. Next, a serial dilution was performed by taking 1.5 mL from the first row and adding it into the adjoining row mixing by pipetting up and down. Two plates were prepared, one was placed at 30 C and the second one, a control plate, was kept at 4 C. Samples were collected at times 0 h, 24 h, 48 h, extracted and analyzed by GC-FID.

The concentration of isoprenol detected in the 24 h time point was divided by the concentration detected at time Oh. A similar isoprenol loss was observed for all tested concentrations and, averaging these values, a 43% loss was determined. We applied the correction for evaporation only at the 48 h timepoint since isoprenol is expected to be produced in the stationary phase.

RBS Mutant pedF-pyrF AND gRNA Library Test to Find Enriched gRNAs

First, dCpf1 was integrated into the genome of the desired Pseudomonas putida strains by allelic exchange to maximize the activity of the gRNA-guided endonuclease. Then PP_1815 (pyrF) was deleted in the same strains by recombineering/aCpf1 to generate mutants that lost the ability to grow in M9 minimal medium without uracil supplementation. The auxotrophy of these strains was tested by growing them first on LB and inoculating them in 5 mL of M9 medium in test tubes after washing them with 10% glycerol. Then, the pedF-pyrF RBS library was transformed in the strains by a standard electroporation protocol plating them on LB gentamicin uracil agar plates. After two days, single colonies were picked and inoculated in 100 μL LB gentamicin in 96-well plates and incubated overnight at 30 C with 40% humidity and no shaking. These strains were then inoculated in three different 96 well plates containing 100 μL of M9 glucose, M9 glucose crystal violet and M9 glucose crystal violet 1.2 g/L isoprenol, gentamicin was not needed because expressing pyrF is the marker needed to grow on these conditions. OD₆₀₀ values were checked and clones that showed the right phenotype (growth M9 cv isoprenol>M9 cv>M9 glu) were inoculated into 5 mL LB gentamicin uracil liquid cultures in test tubes. These strains were genotyped to amplify the RBS by OneTaq PCR (ABC) and sequenced to verify the acquisition of a new RBS sequence. The plasmids were also miniprepped and transformed into the same dCpflint del-pyrF strains.

Triparental conjugation was then performed to introduce the gRNA library in these Pseudomonas putida strains using two Escherichia coli strains; the first one harboring the gRNA library (kanR) and the second one carrying the mobile elements for conjugation. The three strains were grown on LB (indicate gentamicin needed pedF-pyrf and kan for gRNA library), washed two times with 10% glycerol and spotted on LB plates with a ratio of 1:1:1. The next day, the biomass from the LB spots was taken using 200 μL tips and it was resuspended in 500 μL of 10% glycerol. ABC uL were inoculated in 1.5 mL M9 medium kanamycin+/−CV in 24-deep well plates. The lack of growth of both Escherichia coli strains was tested by inoculating them from LB saturated cultures to the same M9 medium with a high starting OD₆₀₀. When the strains reached saturation, 24 h post-inoculation, 1 mL samples were taken and saved −80 C and 30 μL were boiled and the gRNA were amplified by OneTaq PCR. Biomass from the LB conjugation spots was also amplified and sequenced to verify the diversity of the initial distribution of gRNAs.

Isoprenol Biosensor Activity Assay. Standard Assay and the Stationary Phase Assay.

Strains were cultivated on 5 mL LB in test tubes with the indicated antibiotics if harboring any plasmids. The next day, the saturated cultures (uL?) were inoculated in 2 mL M9 minimal medium in 24-deep well plates containing the desired alcohol concentrations. 100 μL samples were taken after 24 and 48 hours to read mCherry absorbance in black opaque 96-well plates at excitation of 587 nm and emission of 610 nm. For stationary phase assay strains were allowed to grow for 24 hours before adding the indicated alcohols.

Strains used are listed in Table 1.

TABLE 1

| | | | |
|---|---|---|---|
| | | Relevant | |
| Strain | Genotype | Figure | Reference |

| | | |
|---|---|---|
| JBEI-13809 | Pseudomonas putida KT2440 wild type | ATCC-47054, |
| | prototroph CmR AmpR | Nieto et al |
| | | 1990 |
| TEAM-2431 | KT2440 P_{J23110}-PP_2666, PP_2665 | This study |
| TEAM-2555 | KT2440 P_{J23110}-PP_2666, PP_2665 | This study |
| | PP_5402int::P_{pedF}-RBSopt-mCherry | |
| TEAM-2583 | KT2440 P_{J23110}-PP_2666, PP_2665 | This study |
| | PP_5402int::P_{pedF}-RBSopt-mCherry | |
| | PP_5322int::eilR; P_{JEx1}-RBS-mvaS, RBS-mvaE | |
| TEAM-2595 | KT2440 P_{J23110}-PP_2666, PP_2665 | This study |
| | PP_5402int::P_{pedF}-RBSopt-mCherry | |
| | PP_5322int::eilR; P_{JEx1}-RBS-mvaS, RBS-mvaE | |
| | PP_0871int::P_{trc}-RBS-mkMM, RBS- | |
| | pmdHKQ, RBS-aphA | |
| TEAM-2632 | KT2440 P_{J23110}-PP_2666, PP_2665 | This study |
| | PP_5402int::P_{pedF}-RBSopt-mCherry | |
| | PP_5322int::eilR; P_{JEx1}-RBS-mvaS, RBS-mvaE | |
| | PP_0871int::P_{trc}-RBS-mkMM, RBS- | |
| | pmdHKQ, RBS-aphA PP_3159::P_{pedF}-RBSopt- | |
| | mCherry | |
| TEAM-2650 | KT2440 P_{J23110}-PP_2666, PP_2665 | This study |
| | PP_5402int::P_{pedF}-RBSopt-mCherry | |
| | PP_5322int::eilR; P_{JEx1}-RBS-mvaS, RBS-mvaE | |
| | PP_0871int::P_{trc}-RBS-mkMM, RBS- | |
| | pmdHKQ, RBS-aphA PP_3159::P_{pedF}-RBSopt- | |
| | mCherry ΔPP_2664 | |
| TEAM-2669 | KT2440 P_{J23110}-PP_2666, PP_2665 | This study |
| | PP_5402int::P_{pedF}-RBSopt-mCherry | |
| | PP_5322int::eilR; P_{JEx1}-RBS-mvaS, RBS-mvaE | |
| | PP_0871int::P_{trc}-RBS-mkMM, RBS- | |
| | pmdHKQ, RBS-aphA PP_3159::P_{pedF}-RBSopt- | |
| | mCherry ΔPP_2664 ΔPP_2675 | |
| TEAM-2777 | KT2440 P_{J23110}-PP_2666, PP_2665 | This study |
| | PP_5402int::P_{pedF}-RBSopt-mCherry | |
| | PP_5322int::eilR; P_{JEx1}-RBS-mvaS, RBS-mvaE | |
| | PP_0871int::P_{trc}-RBS-mkMM, RBS- | |
| | pmdHKQ, RBS-aphA PP_3159::P_{pedF}-RBSopt- | |
| | mCherry ΔPP_2664 ΔPP_2675 P_{J3119}- | |
| | PP_1697 | |
| TEAM-3174 | KT2440 P_{J23110}-PP_2666, PP_2665 | This study |
| | PP_5402int::P_{pedF}-RBSopt-mCherry | |
| | PP_5322int::eilR; P_{JEx1}-RBS-mvaS,RBS-mvaE | |
| | PP_0871int::P_{trc}-RBS-mkMM, RBS- | |
| | pmdHKQ, RBS-aphA PP_3159::P_{pedF}-RBSopt- | |
| | mCherry ΔPP_2664 ΔPP_2675 P_{J3119}- | |
| | PP_1697 ΔPP_2428 ΔPP_4622 | |
| | ΔPP_3450/mvaB ΔPP_4373/fleQ ΔPP_2710 | |
| | PP_5454int::P_{JEx1}-mvaS | |
| TEAM-3185 | KT2440 P_{J23110}-PP_2666, PP_2665 | This study |
| | PP_5402int::P_{pedF}-RBSopt-mCherry | |
| | PP_5322int::eilR; P_{JEx1}-RBS-mvaS, RBS-mvaE | |
| | PP_0871int::P_{trc}-RBS-mkMM, RBS- | |
| | pmdHKQ, RBS-aphA PP_3159::P_{pedF}-RBSopt- | |
| | mCherry ΔPP_2664 ΔPP_2675 P_{J3119}- | |
| | PP_1697 ΔPP_2428 ΔPP_4622 | |
| | ΔPP_3450/mvaB ΔPP_4373/fleQ ΔPP_2074 | |
| gRNA | ΔPP_2664 | |
| plasmid | | |
| gRNA | ΔPP_2675 | |
| plasmid | | |
| gRNA | PP_1697 promoter | |
| plasmid | | |
| gRNA | PP_2428 | |
| plasmid | | |
| gRNA | PP_4622 | |
| plasmid | | |
| gRNA | PP_3450 mvaB | |
| plasmid | | |
| gRNA | PP_4373/fleQ | Eng and |
| plasmid | | Banerjee Cell |
| | | Reports 2023 |
| gRNA | PP_2074 | |
| plasmid | | |
| gRNA | PP_2710 | |
| plasmid | | |
| Biosensor | pTE518 | |
| plasmid | | |
| Biosensor | pTE520 | |
| integration | | |
| cassette | | |
| pSPIN | pTE709 | |
| integration | | |
| plasmid | | |
| pSPIN | pTE1021 | |
| integration | | |
| plasmid | | |
| | pBAD-PP | |

### Example 2

### An Isoprenol Biosensor for High Throughput Titer Quantification in Pseudomonas putida

Objective: Build a high throughput (HT) fluorescence-based biosensor to monitor isopentenol (isoprenol) production.

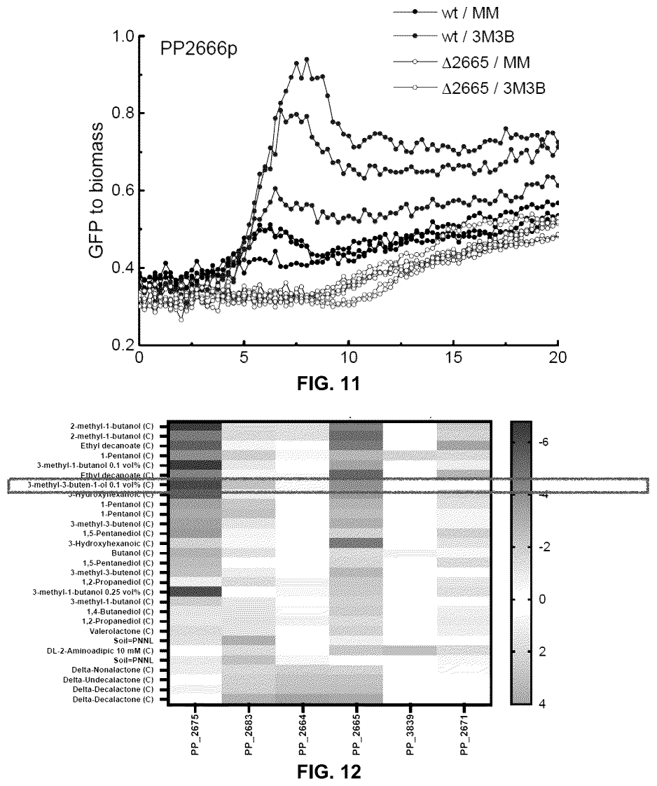

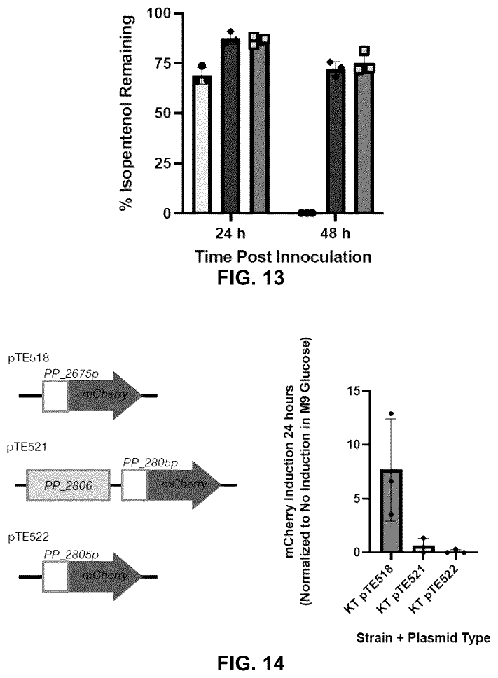

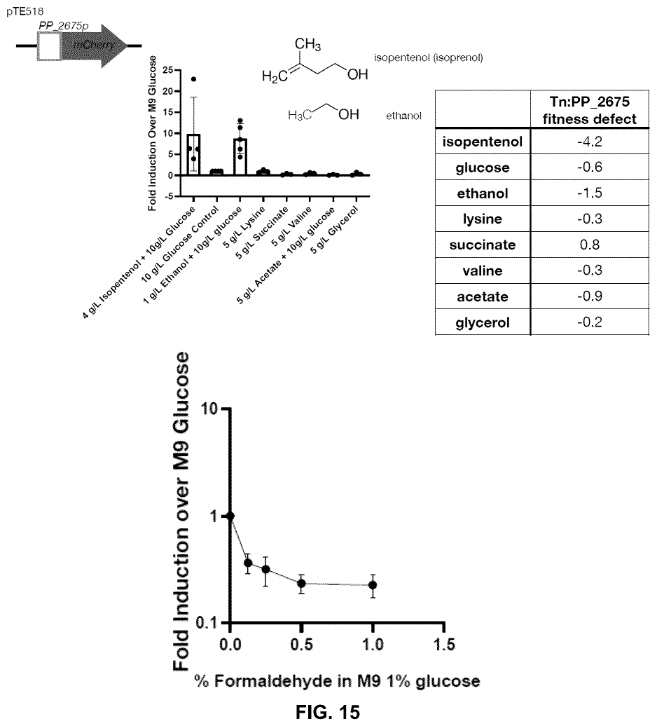

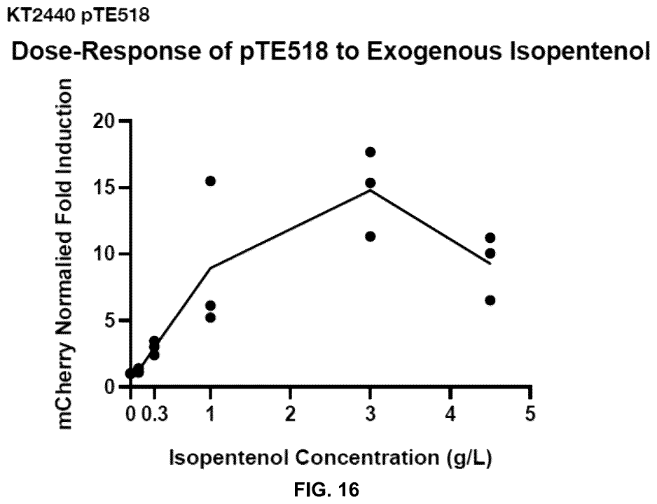

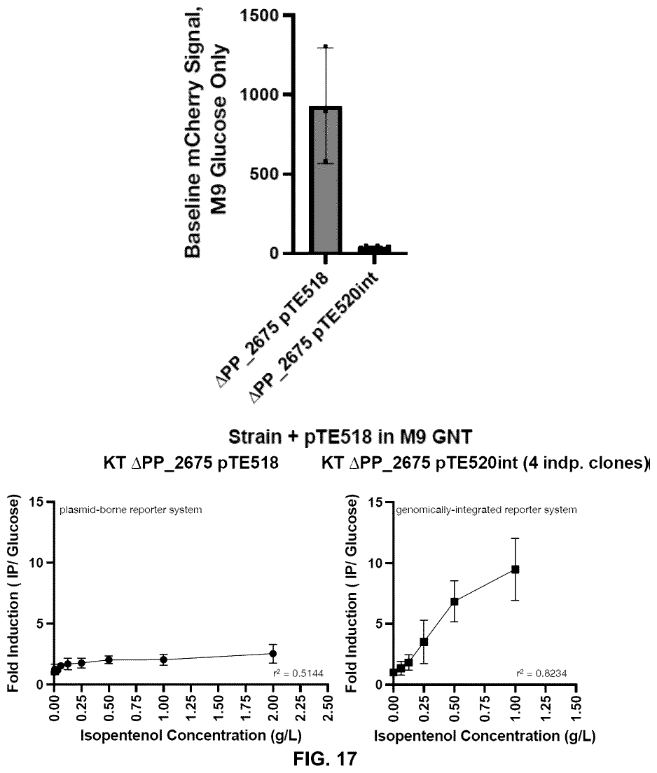

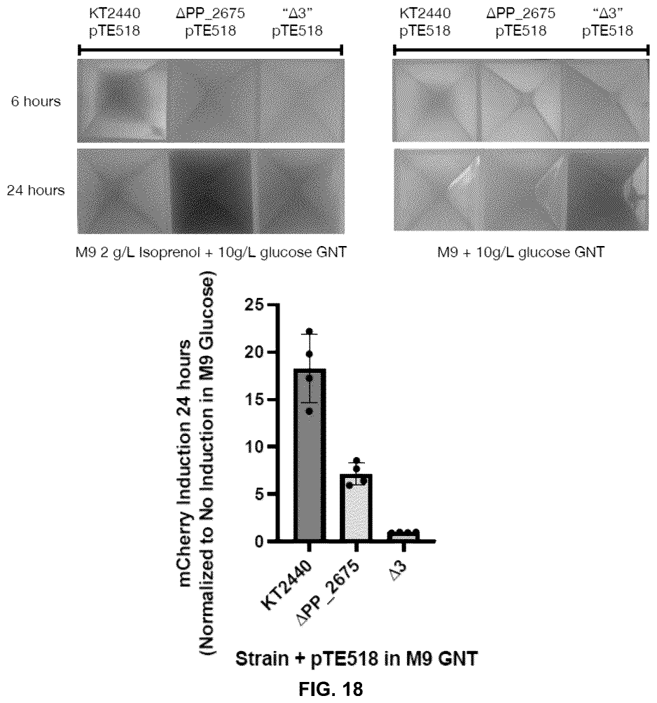

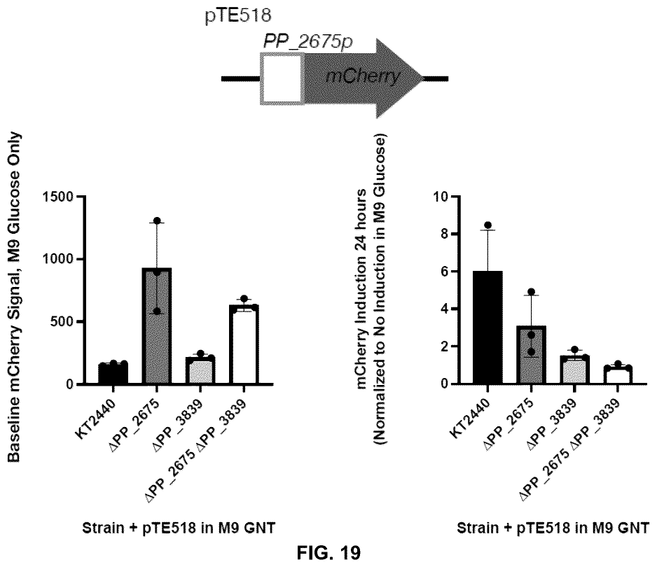

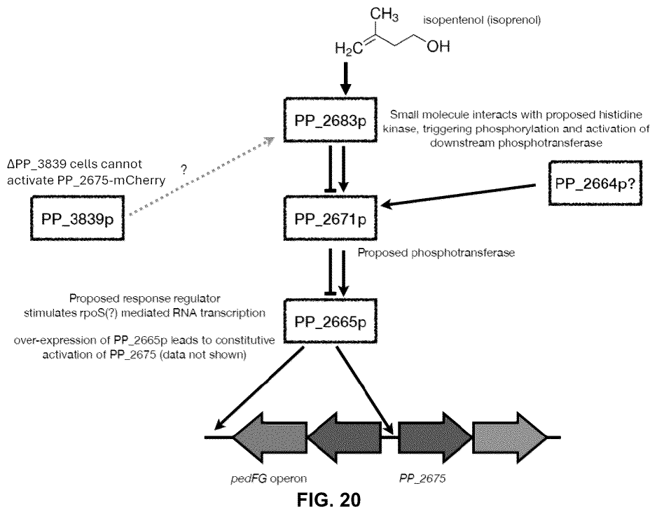

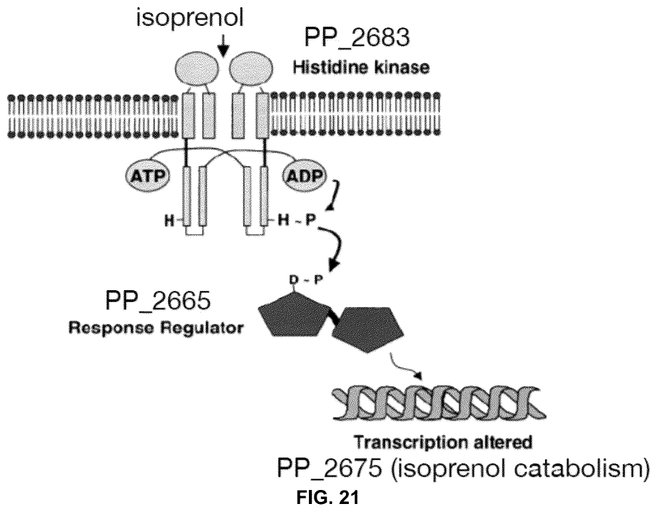

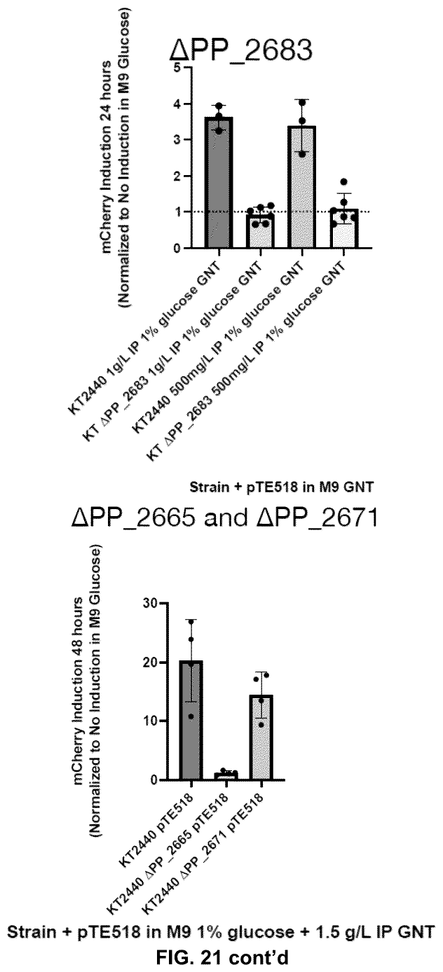

The goal of the project was to identify DNA-responsive elements which respond to isopentenol. At the project start, we tried building a few isoprenol responsive promoters but the signal/noise ratio had room for improvement. A maximum 2.25×GFP induction (n=1 out of 3) is observed, wherein the signal dropped to 1.5× after 24 hours. This promoter drives expression of a potential isoprenol-sensitive response regulator PP_2665. An E. coli RBS driving GFP was chosen rather than a P. putida RBS (FIG. 11). Two potential DNA promoter sequences were identified: PP_2675 and PP_2805. Transposon mutants in pp 2675 are compromised in their ability to grow on isopentenol (Thompson et al 2020) and ΔPP_2675 strains do not catabolize isopentenol (Russel Menchavez, unpublished) (FIG. 12). Short-term treatment of P. putida with exogenous isopentenol induces 100× expression of PP_2805 (Hyungyu Lim, unpublished) (FIG. 13). Three promoter-mCherry constructs are generated, but only pTE518 using a PP_2675 promoter showed a response to exogenous isopentenol. See FIG. 14. pPP_2675 likely responds to alcohols of varied chain lengths but not other common overflow metabolites (FIG. 15). The PP_2675 promoter shows a dose-dependent response to exogenous isoprenol (FIG. 16). Integrating the IP Biosensor Both Reduces Baseline mCherry Fluorescence and Increases Dynamic Response Range (KT ΔPP_2675 pTE518 and KT ΔPP_2675 pTE520int, 4 indp. clones) (FIG. 17). KT2440 ΔPP_2675 has comparable isopentenol induction to WT, but the triple mutant (ΔPP_2675 ΔPP_3839 ΔPP_4064-4067) shows constitutive activation (FIG. 18). PP_2675p represses its own baseline expression; PP_3839p enhances PP_2675 induction (FIG. 19). Herein is proposed a HK/RR Signaling Cascade Pathway to express PP_2675 (FIG. 20). Small molecule interacts with proposed histidine kinase, triggering phosphorylation and activation of downstream phosphotransferase. ΔPP_3839 cells cannot activate PP_2675-mCherry. Over-expression of PP_2665p leads to constitutive activation of PP_2675 (data not shown).

### Example 3

### A Diol Biosensor in Pseudomonas putida

The experiments above were repeated but using diols instead of isoprenol. A plate reader was used instead of the flow cytometer to repeat the experiment of. The fold response is lower than that obtained for isoprenol. Such results are expected for a plate reader, but is more generally accessible as a method since plate readers are much more common. The diols tested were 1,5 pentanediol and 1,2 propanediol. All concentrations tested were at 11.6 mM, since we use 11.6 mM isoprenol for the original characterization.

The fold activation with 11.6 mM 1,2-propanediol is about 22-fold. The response is somewhat variable between biological replicates, and there is a bimodal response. 11.6 mM of isoprenol is equal to 1 g/L, so it is an equimolar amount of 1,2-propanediol for comparison. 1,2-propanediol is an industrial commodity chemical. An in vitro assay can be done with pp 2682 including 1,2-propanediol which would be useful due to its clean annotation as a propanediol oxidoreductase.

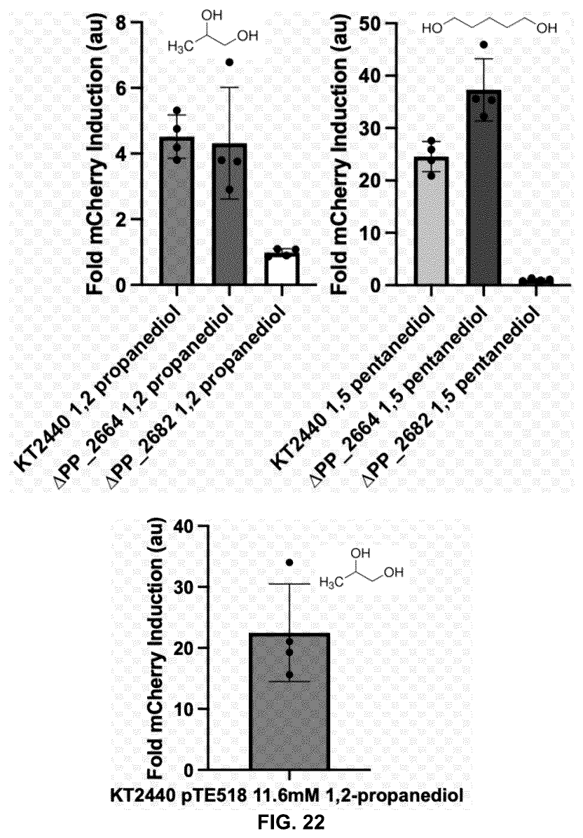

An ML-pipeline is used to reannotate PP_2682, which helps process isoprenol for the biosensor. The new annotation comes back as a 1,2-propanediol oxidoreductase. Using that annotation it is confirmed that the isoprenol biosensor can also quantify 1,2-propanediol There is at least one bioconversion pathway for glucose to 1,2 propanediol described in Corynebacterium glutamicum. In addition to isoprenol, 1,2-propanediol or 1,5-pentanediol also induce the mCherry expression, and KT2440 pTE518 is also able to demonstrate an over 20-fold induction by 1,2-propanediol (FIG. 22).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for increasing the production of a short chain alcohol, comprising: (a) providing a microbial strain comprising a genetic circuit comprising a short chain alcohol biosensor which allows growth of the microbial strain only when the microbial strain produces the short chain alcohol is produced, (b) screening for a cell with increased short chain alcohol production, and (c) optionally culturing or growing the cell to produce the short chain alcohol

2. The method of claim 1, wherein the short chain alcohol is isoprenol or a diol.

3. The method of claim 2, wherein the diol has 3, 4, or 5 carbon atoms.

4. The method of claim 3, wherein the diol is 1,2-propanediol or 1,5-pentanediol
